(19) United States
(12) Patent Application Publication
(10) Pub. No.: US 2012/0071640 A1
(43) Pub. Date: Mar. 22, 2012

(54) Sulfurizing reagents and their use for oligonucleotides synthesis

(75) Inventors: Wieslaw Adam MAZUR, Mason, OH (US); Yigang HE, Ewing, NJ (US); Victor D. SOROKIN, Cincinnati, OH (US)

(73) Assignee: GIRINDUS AMERICA, INC., Cincinnati, OH (US)

(21) Appl. No.: 13/165,918

(22) Filed: Jun. 22, 2011

Related U.S. Application Data
(63) Continuation-in-part of application No. PCT/EP2009/067902, filed on Dec. 23, 2009.
(60) Provisional application No. 61/360,365, filed on Jun. 30, 2010.
(60) Provisional application No. 61/140,391, filed on Dec. 23, 2008.

(51) Int. Cl. C07H 21/00; C07D 207/48; C07H 21/04; C07H 1/00; C07H 21/02; C07C 311/48; C07C 303/40

(52) U.S. Cl. 536/23.1; 560/129; 548/542; 558/276; 560/165; 536/25.3; 536/25.6

(57) ABSTRACT

An oligonucleotide which comprises at least one internucleotide linkage comprising a P—S—R bond and at least two nucleosides, wherein R corresponds to the formula (I)

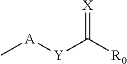

wherein A is a geminally substituted alkylene group, preferably CH₂, X and Y are independently selected from S and O, and R₀ is selected from the group consisting of optionally substituted carbon bonded organic residue, such as in particular optionally substituted alkyl or aryl, SRx, ORx and NRxRy wherein Rx and/or Ry are selected from H and organic residues and at least Rx is a substituent other than H. Another object of the invention is a sulfurizing agent useful for oligonucleotide manufacture and the manufacture thereof. Other nucleotides described comprise at least one internucleotide linkage comprising a P—S—R bond, at least one internucleotide linkage comprising a P—S—R′ bond and at least three nucleosides wherein R′ is an organic residue other than the group R, preferably selected from a group consisting of an aryl group and a heteroaryl group which is bonded to the S-atom through an annular carbon atom.

### CROSS-REFERENCE TO RELATED APPLICATIONS

The present application claims the benefit of U.S. provisional application No. 61/360,365 filed Jun. 30, 2011, and is a continuation-in-part application of International Application No. PCT/EP2009/067902 filed Dec. 23, 2009, which itself claims the benefit of U.S. provisional application No. 61/140,391 filed Dec. 23, 2008, the whole content of all these applications being incorporated herein by reference for all purposes.

### TECHNICAL FIELD

The present invention relates to phosphorothioate oligonucleotides, the preparation thereof using novel sulfurizing reagents, said sulfurizing reagents and the preparation thereof.

### BACKGROUND ART

Oligonucleotides belong to a class of biopharmaceuticals with a great potential for therapies of various diseases including cancer, viral infections and inflammatory disease to name a few. An important approach to advancing oligonucleotides as therapeutics involve modifications of the oligomer backbone to provide, among other things, metabolic resistance, chemical stability and to improve in vivo transport to the site of action. Examples of modified backbone chemistries include: peptide nucleic acids (PNAs) (see Nielsen, Methods Mol. Biol., 208:3-26, 2002), locked nucleic acids (LNAs) (see Petersen & Wengel, Trends Biotechnol., 21(2):74-81, 2003), phosphorothioates (see Eckstein, Antisense Nucleic Acid Drug Dev., 10(2):117-21, 2000), methylphosphonates (see Thiviyanathan et al., Biochemistry, 41(3):827-38, 2002), phosphoramidates (see Gryaznov, Biochem. Biophys. Acta, 1489(1):131-40, 1999; Pruzan et al., Nucleic Acids Res., 30(2):559-68, 2002), thiophosphoramidates (see Gryaznov et al., Nucleosides Nucleotides Nucleic Acids, 20(4-7):401-10, 2001; Herbert et al., Oncogene, 21(4):638-42, 2002). Formation of phosphorothioates belongs to the most useful modifications since the replacement of P═O with P═S moiety makes the oligonucleotides resistant to nucleolytic degradation while retaining in most cases the biological properties of natural oligomers.

Phosphorothioates can be formed by oxidative sulfurization (Oligonucleotide synthesis, methods and applications, P. Herdewijn Methods in Molecular Biology, volume 288, Chapter 4, 51-63). There are basically two approaches to making phosphorothioates depend upon the nature of phosphorous esters used for this reaction and the expected products. One of them involves introduction of the unsubstituted sulfur atom to phosphorus by means of, for example, elemental sulfur, dibenzoyl tetrasulfide, 3-H-1,2-benzodithiol-3-one 1,1-dioxide (also known as Beaucage reagent, (Iyer et al., J. Org. Chem. 55, 4693-4699 (1990)), tetraethylthiuram disulfide (TETD), dimethylthiuram disulfide (DTD), phenylacetyl disulfide (PADS) and bis(O,O-diisopropoxy phosphinothioyl) disulfide (known as Stec's reagent. These reactions are mostly used in the automated synthesis of oligonucleotides on solid support by the phosphoramidite method and comprise the oxidative sulfurization of phosphorus triesters formed during the elongation reaction of oligomers

A second approach to making oligomeric phosphorothioates is used with the H-phosphonate method and involves a reaction between H-phosphonate diester and a sulfur transfer reagent in which the sulfur atom, bearing an aliphatic or aromatic substituent, is transferred to phosphorus. The auxiliary substituent at sulfur serves the role of a protecting group during the synthetic operation and usually is cleaved at the final stage of oligonucleotide preparation. This method is particularly suitable for the synthesis of oligonucleotides in solution.

In contrast to large selection of reagents available for introducing the unsubstituted sulfur atom to phosphorus esters, the spectrum of groups allowing for sulfurization of H-phosphonate esters with the protected sulfur is limited (e.g. Dreef, et al. Synlett, 481-483, 1990, U.S. Pat. No. 6,506,894). Practically, only the cyanoethylsulfide group has been used extensively in this reaction during the solution synthesis of oligonucleotides with chromatographic purification at each step. A critical problem in the solution synthesis of oligonucleotides concerns the necessity to obtain high substrate conversions with excellent specificity at each synthetic step giving high purity products in a form that facilitates simple purification, in particular avoiding chromatography. Given the lack of methods allowing for economical solution phase synthesis, the solution phase technology does not seem to be currently used for commercial scale oligonucleotide synthesis. The invention now discloses novel sulfurizing reagents, a process for their manufacture and their use in the economical and convenient synthesis and purification of phosphorothioate oligonucleotides notably in solution.

### SUMMARY OF INVENTION

The present invention relates in particular to the invention described in the appended claims. The invention also relates to processes and reagents substantially described in the present specification, in particular in the examples.

The invention has a number of advantages over existing methods of P—S linkage formation, in particular in the synthesis of oligonucleotides carried out preferably via the H-phosphonate method. For example, the residue R transferred e.g. to an oligonucleotide with the novel reagent can facilitate crystallization or precipitation of oligonucleotides, allowing for simple purification of the products with minimum or no chromatography. It has been found out that the oligonucleotides having from two to at least sixteen nucleotide units made with this method do not necessarily have to be purified by chromatography until after the final deprotection of the required oligonucleotide. The intermediate oligomers can be obtained pure enough for optional deprotections at the 5′-and 3′-positions and further coupling of these crude deprotected materials to higher oligonucleotides, if desired. In another advantage, the disclosed method provides an access to a variety of sulfurizing reagents which can be used to modify the properties of formed oligonucleotides with respect to maximizing the efficiency of simple, chromatography-free purifications. Still another advantage of the method according to the invention is that a simple cleavage of for example the sulfur-protecting acyloxymethylene group RC(O)—OCH₂ can be easily accomplished under mild conditions, for example, with primary or secondary or hindered amines e.g. n-propylamine or tert-butylamine. Upon the optional treatment with an amine, a spontaneous cleavage, including the sulfur-methylene bond occurs thus allowing for the clean formation of P═S bond. The cleavage products can be easily removed from the products by solvent or aqueous wash. In a particular aspect, the cleavage of the sulfur-protecting group in accordance with the present invention, for example the sulfur-protecting acyloxymethylene group RC(O)—OCH₂ can be carried out selectively whereby for example the cleavage of other optionally present sulfur-protecting groups, such as in particular suitable aryl groups can be prevented. The presence of different protective groups on different sulfur atoms of an oligonucleotide having at least 2 sulfurized internucleotide bonds, which protective groups have substantially different reactivity, allows for selective deprotection so as to be able to selectively create sulfurized or oxygenated internucleotide bonds. This aspect of the invention allows to prepare an oligonucleotide containing both phosphodiester and phosphothioate diester internucleotide linkages in the same molecule. The stability characteristics for example of the acyloxymethylene group e.g. under basic non-nucleophilic conditions allow for selective deprotection reactions along the synthesis pathways and hence greater flexibility of synthesis schemes, for example by preventing the cleavage of nucleobase protection groups.

An important factor in developing an economical process for the synthesis of oligonucleotides, especially in solution, is the purity of the transformation product at each step of oligonucleotide chain elongation. Even though the process according to the invention secures high yields and purity of the products, each elongation cycle comprises generally three steps and it is advantageous to remove even small amounts of impurities which would otherwise accumulate along the way. Because of large number of steps, the use of chromatography at each step may not be economically feasible in the practical large scale oligonucleotide synthesis. Therefore, we also disclose a chromatography-free methodology for the purification of oligonucleotides formed during the chain elongation process.

### DETAILED DESCRIPTION

A first particular object of this invention is to provide oligonucleotides which comprise at least one internucleotide linkage comprising a P—S—R bond and at least two nucleosides, wherein R corresponds to the formula (I)

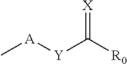

wherein A is a geminally substituted alkylene group, preferably CH₂, X and Y are independently selected from S and O, and R₀ is selected from the group consisting of optionally substituted carbon bonded organic residue, such as in particular optionally substituted alkyl or aryl, SRx, ORx and NRxRy wherein Rx and Ry are selected from H and organic residues and at least Rx is a substituent other than H.

A second particular object of this invention is to provide oligonucleotides which comprise at least one internucleotide linkage comprising a P—S—R bond, at least one internucleotide linkage comprising a P—S—R′ bond and at least three nucleosides, wherein R corresponds to the formula

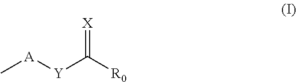

wherein A is a geminally substituted alkylene group, preferably CH₂, X and Y are independently selected from S and O, and R₀ is selected from the group consisting of optionally substituted carbon bonded organic residue, such as in particular optionally substituted alkyl or aryl, SRx, ORx and NRxRy wherein Rx and Ry are selected from H and organic residues and at least Rx is a substituent other than H, and
wherein R′ is an organic residue other than the group R, preferably selected from a group consisting of an aryl group and a heteroaryl group which is bonded to the S-atom through an annular carbon atom.

It has been found that the oligonucleotides according to the invention are valuable synthesis intermediates for synthesis of P-sulfurized oligonucleotides which have advantageous properties as to their solubility characteristics thus allowing for efficient purification which can be effectively accomplished, for example, by a combination of precipitation and extraction techniques. The oligonucleotides according to the invention are also believed to be effective as pro-drug, capable to release a phosphorothioate oligonucleotide in vivo, by cleavage of the R-group in the human body or in the body of an animal.

The following definitions relate to the oligonucleotides of the present invention which are provided according to the first particular object of the invention and to the oligonucleotides of the present invention which are provided according to the second particular object of the invention.

The term “oligonucleotide”, in the frame of the present invention, denotes in particular an oligomer of nucleoside monomeric units comprising sugar units connected to nucleobases, said nucleoside monomeric units being connected by internucleotide bonds. An “internucleotide bond” refers in particular to a chemical linkage between two nucleoside moieties, such as the phosphodiester linkage typically present in nucleic acids found in nature, or other linkages typically present in synthetic nucleic acids and nucleic acid analogues. Such internucleotide bond may for example include a phospho or phosphite group, and may include linkages where one or more oxygen atoms of the phospho or phosphite group are either modified with a substituent or replaced with another atom, e.g., a sulfur atom, or the nitrogen atom of a mono- or di-alkyl amino group. Typical internucleotide bonds are diesters of phosphoric acid or its derivatives, for example phosphates, thiophosphates, dithiophosphate, phosphoramidates, thio phosphoramidates.

The term “nucleoside” is understood to denote in particular a compound consisting of a nucleobase connected to a sugar. Sugars include, but are not limited to, furanose ring such as ribose, 2′-deoxyribose and non-furanose ring such as cyclohexenyl, anhydrohexitol, morpholino. The modifications, substitutions and positions indicated hereinafter of the sugar included in the nucleoside are discussed with reference to a furanose ring, but the same modifications and positions also apply to analogous positions of other sugar rings. The sugar may be additionally modified. As non limitative examples of the modifications of the sugar mention can be notably made of modifications at e.g. the 2′-or 3′-position, in particular 2′-position of a furanosyl sugar ring including for instance hydrogen; hydroxy; alkoxy such as methoxy, ethoxy, allyloxy, isopropoxy, butoxy, isobutoxy, methoxyethyl, alkoxy, phenoxy; azido; amino; alkylamino; fluoro; chloro and bromo; 2′-4′-and 3′-4′-linked furanosyl sugar ring modifications, modifications in the furanosyl sugar ring including for instance substitutions for ring 4′-0 by S, CH₂, NR, CHF or CF₂.

The term “nucleobase” is understood to denote in particular a nitrogen-containing heterocyclic moiety capable of pairing with a, in particular complementary, nucleobase or nucleobase analog. Typical nucleobases are the naturally occurring nucleobases including the purine bases adenine (A) and guanine (G), and the pyrimidine bases thymine (T), cytosine (C) and uracil (U), and modified nucleobases including other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine, and fluorinated bases. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Other potentially suitable bases include universal bases, hydrophobic bases, promiscuous bases and size-expanded bases.

“Oligonucleotide” typically refers to a nucleoside subunit polymer having from about 2 to about 50 contiguous subunits. The nucleoside subunits can be joined by a variety of intersubunit linkages. Further, “oligonucleotides” includes modifications, known to one skilled in the art, to the sugar backbone (e.g., phosphoramidate, phosphorodithioate), the sugar (e.g., 2′ substitutions such as 2′-F, 2′-OMe), the base, and the 3′ and 5′ termini. Typically, in this embodiment of the invention the oligonucleotide comprises from 2 to 30 nucleotides. In different embodiments of this invention, the oligonucleotide contains nucleosides selected from ribonucleosides, 2′-deoxyribonucleosides, 2′-substituted ribonucleosides, 2′-4′-locked-ribonucleosides, 3′-amino-ribonucleosides, 3′-amino-2′-deoxyribonucleosides.

The term “organic residue” is intended to denote in particular linear or branched alkyl or alkylene groups which may contain hetero atoms, such as in particular boron, silicon, nitrogen, oxygen or sulfur atoms and halogen atoms, cycloalkyl groups, heterocycles and aromatic systems. The organic residue may contain double or triple bonds and functional groups.

The organic residue comprises at least 1 carbon atom. It often comprises at least 2 carbon atoms. It preferably comprises at least 3 carbon atoms. More particularly preferably, it comprises at least 5 carbon atoms. The organic residue generally comprises at most 100 carbon atoms. It often comprises at most 50 carbon atoms. It preferably comprises at most 40 carbon atoms. More particularly preferably, it comprises at most 30 carbon atoms.

The term “alkylene group” or “cycloalkylene group” is intended to denote in particular the divalent radicals derived from the alkyl or cycloalkyl groups as defined above.

When the organic residue contains one or optionally more double bonds, it is often chosen from an alkenyl or cycloalkenyl group comprising from 2 to 20 carbon atoms, preferably 2, 3, 4, 5, 6, 7, 8, 9 or 10 carbon atoms. Specific examples of such groups are vinyl, 1-allyl, 2-allyl, n-but-2-enyl, isobutenyl, 1,3-butadienyl, cyclopentenyl, cyclohexenyl and styryl.

When the organic residue contains one or optionally more triple bonds, it is often chosen from an alkinyl group comprising from 2 to 20 carbon atoms, preferably 2, 3, 4, 5, 6, 7, 8, 9 or 10 carbon atoms. Specific examples of such groups are ethinyl, 1-propinyl, 2-propinyl, n-but-2-inyl and 2-phenylethinyl.

When the organic residue contains one or optionally more aromatic systems, it is often an aryl or an alkylaryl group comprising from 6 to 24 carbon atoms, preferably from 6 to 12 carbon atoms. Specific examples of such groups are phenyl, 1-tolyl, 2-tolyl, 3-tolyl, xylyl, 1-naphthyl and 2-naphthyl.

The term “heterocycle” is intended to denote in particular a cyclic system comprising at least one saturated or unsaturated ring made up of 3, 4, 5, 6, 7 or 8 atoms, at least one of which is a hetero atom. The hetero atom is often chosen from B, N, O, Si, P and S. It is more often chosen from N, O and S.

Specific examples of such heterocycles are aziridine, azetidine, pyrrolidine, piperidine, morpholine, 1,2,3,4-tetrahydroquinoline, 1,2,3,4-tetrahydroiso-quinoline, perhydroquinoline, perhydroisoquinoline, isoxazolidine, pyrazoline, imidazoline, thiazoline, tetrahydrofuran, tetrahydrothiophene, pyran, tetra-hydropyran and dioxane.

The organic residues as defined above may be unsubstituted or substituted with functional groups.

The term “functional group” is intended to denote in particular a substituent comprising or consisting of a hetero atom. The hetero atom is often chosen from B, N, O, Al, Si, P, S, Sn, As and Se and the halogens. It is more often chosen from N, O, S and halogen, in particular halogen.

The functional group generally comprises 1, 2, 3, 4, 5 or 6 atoms.

By way of functional groups, mention may, for example, be made of halogens, a hydroxyl group, an alkoxy group, a mercapto group, an amino group, a nitro group, a carbonyl group, an acyl group, an optionally esterified carboxyl group, a carboxamide group, a urea group, a urethane group and the thiol derivatives of the abovementioned groups containing a carbonyl group, phosphine, phosphonate or phosphate groups, a sulphoxide group, a sulphone group and a sulphonate group.

The term “aryl group” is intended to denote in particular an aromatic carbocyclic system comprising from 6 to 24 carbon atoms, preferably from 6 to 12 carbon atoms. The aryl group may be unsubstituted or substituted for example, with aryl or heteroaryl, alkyl groups, cycloalkyl, or functional groups.

The term “heteroaryl” is intended to denote in particular an aromatic carbocyclic system comprising from 5 to 24 atoms, preferably from 5 to 12 atoms, at least one of which is a hetero atom. The hetero atom is often chosen from B, N, O, Si, P and S. It is more often chosen from N, O and S.

The term “alkyl group” is intended to denote in particular a linear or branched alkyl substituent comprising from 1 to 20 carbon atoms, preferably 1, 2, 3, 4, 5, 6, 7, 8, 9 or 10 carbon atoms. Specific examples of such substituents are methyl, ethyl, n-propyl, isopropyl, n-butyl, isobutyl, tert-butyl, n-pentyl, isopentyl, n-hexyl, 2-hexyl, n-heptyl, n-octyl and benzyl.

The term “cycloalkyl group” is intended to denote in particular a substituent comprising at least one saturated carbocycle containing 3 to 10 carbon atoms, preferably 5, 6 or 7 carbon atoms. Specific examples of such substituents are cyclopropyl, cyclobutyl, cyclopentyl, cyclohexyl and cycloheptyl.

In some oligonucleotides of the invention, R is selected from a methyleneacyloxy group, a methylene carbonate group and a methylene carbamate group.

In some oligonucleotides of the invention, especially those provided according to the second particular object of the invention, R is selected from a methyleneacyloxy group, a methylene carbonate group and a methylene carbamate group; and R′ is an unsubstituted or substituted phenyl group, in particular a 4-halophenyl group or a 4-alkylphenyl group, for example a 4-(C₁-C₄-alkyl)phenyl group; R′ is especially a 4-chlorophenyl group.

When R is a methyleneacyloxy group, it corresponds preferably to formula —CH₂—O—C(O)—R₀ wherein R₀ is a C1-C20, saturated, unsaturated, heterocyclic or aromatic, hydrocarbon residue. When R₀ is a saturated hydrocarbon residue, it is preferably selected from linear, branched or cyclic alkyl residues. R₀ can for example be selected from lower alkyl or cycloalkyl (C1-C7) residues. Particular saturated hydrocarbon residues are selected from methyl, ethyl, n-propyl, isopropyl, n-butyl, sec-butyl, tert. butyl, cyclopentyl and cyclohexyl. A methyl, ethyl or n-propyl group is preferred. An ethyl group is more particularly preferred. When R₀ is an aromatic residue, it is suitably selected from aromatic systems having from 6 to 14 carbon atoms. Particular aromatic residues are selected from phenyl and naphthyl groups which can be substituted, for example, by aryl or heteroaryl, alkyl, cycloalkyl, heterocycle or heterosubstitutents such as halogens, amines, ethers, carboxylates, nitro, thiols, sulfonic and sulfones. A phenyl group is preferred. When R₀ is a heterocyclic residue, it is often selected from heterocycles containing at least one annular N, O or S atom which are bonded to the carbonyl group through an annular carbon atom. Particular examples of such heterocyclic residues include pyridine and furan.

In a particular aspect, the oligonucleotide comprises at least two internucleotide linkages comprising a P—S—R bond and at least three nucleotides, wherein R is a methyleneacyloxy group as described herein.

When R is a methylene carbamate group, it preferably corresponds to formula —CH₂—O—C(O)—NRxRy wherein Rₓ and Ry are independently selected from alkyl or (hetero)aryl. Preferably Rx and/or Ry are alkyl groups. In this case Rx and/or Ry can for example be selected from lower alkyl or cycloalkyl (C1-C7) residues. Particular alkyl groups are selected from methyl, ethyl, n-propyl, isopropyl, n-butyl, sec-butyl, tert butyl, cyclopentyl and cyclohexyl. A methyl, ethyl or n-propyl group is preferred. In a particular preferred aspect, Rx and Ry in the methylene carbamate group are both alkyl groups, in particular as described herein before. A N,N-dimethyl or N,N-diethyl group is more particularly preferred. In another aspect of this embodiment Rₓ and Ry form together a 3 to 8 membered ring optionally containing an additional annular heteroatom selected from O, N and S. Particular examples include a N-piperidyl or an N-pyrrolidyl group.

When R is a methylene carbonate group, it corresponds preferably to formula —CH₂—O—C(O)ORx wherein Rx is selected from optionally substituted alkyl, cycloalkyl and (hetero)aryl groups. Preferably, Rx is an alkyl group. In this case Rx can for example be selected from lower alkyl or cycloalkyl (C1-C7) residues. Particular alkyl groups are selected from methyl, ethyl, n-propyl, isopropyl, n-butyl, sec-butyl, tert butyl, cyclopentyl and cyclohexyl. A methyl, ethyl or n-propyl group is preferred. An ethyl group is more particularly preferred. When Rx is an aryl group, it is suitably selected from aromatic systems having from 6 to 14 carbon atoms. Particular aromatic residues are selected from phenyl and naphthyl groups. A phenyl group is preferred. When Rx is a heterocyclic residue, it is often selected from heterocycles containing at least one annular N, O or S atom which are bonded to the oxycarbonyl group through an annular carbon atom. Particular examples of such heterocyclic residues include pyridine and furan.

Rx is preferably selected from lower alkyl or cyclolkyl (C1-C7), phenyl including substituted phenyl and naphthyl groups when R is selected from a methyleneacyloxy group, a methylene carbonate group and a methylene carbamate group.

It is understood that the definitions and preferences of substituents Rx, Ry and R₀, given herein before for the case when R is selected from methyleneacyloxy group, a methylene carbonate group and a methylene carbamate group equally apply to the corresponding thioanalogues wherein X and/or Y in formula (I) are sulfur. It is also understood that the mentioned substituents may be optionally substituted, for example by halogen or alkoxy substituents or they may be modified, for example by inclusion of catenary heteroatoms, in particular oxygen into an alkyl chain.

Thus, to summarize preferred embodiments of the nucleotides provided by the second particular aspect of the invention which comprise at least one internucleotide linkage comprising a P—S—R bond and at least one internucleotide linkage comprising a P—S—R′ bond and at least three nucleotides:
R is preferably is selected from a methyleneacyloxy group, a methylene carbonate group and a methylene carbamate group; and R′ is an unsubstituted or substituted phenyl group, preferably a 4-halophenyl group or a 4-alkylphenyl group, more preferably 4-chlorophenyl group.
In one embodiment, R preferably corresponds to formula —CH₂—O—C(O)—R₀ wherein R₀ is a C1-C20, saturated, unsaturated, heterocyclic or aromatic, hydrocarbon residue.
In one embodiment, R corresponds to a methylene carbamate group of formula —CH₂—O—C(O)—NRxRy wherein Rₓ and Ry are independently selected from alkyl or (hetero)aryl preferably Rx and Ry are alkyl or Rₓ and Ry form together a 3 to 8 membered ring optionally containing an additional annular heteroatom selected from O, N and S.
In one embodiment, R corresponds to a methylene carbonate group of formula —CH₂—O—C(O)ORx wherein Rₓ is selected from optionally substituted alkyl cycloalkyl and (hetero)aryl groups.
Rₓ is preferably selected from lower alkyl or cycloalkyl (C1-C7), phenyl including substituted phenyl and naphthyl groups.
The oligonucleotide preferably comprises from 2 to 30 nucleotides.
The oligonucleotide preferably contains nucleosides selected from ribonucleosides, 2′-deoxyribonucleosides, 2′-substituted ribonucleosides, 2′-4′-locked-ribonucleosides, 3′-amino-ribonucleosides, 3′-amino-2′-deoxyribonucleosides.
The oligonucleotide as a prodrug.

The still more preferred embodiments are those described above. A third particular object of this invention relates to a sulfurizing agent of formula R″—S—R wherein R is as defined here before in the context of the oligonucleotide according to the invention and R″ is a leaving group. Accordingly, R corresponds to the formula (I)

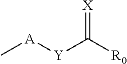

wherein A is a geminally substituted alkylene group, preferably CH₂, X and Y are independently selected from S and O, and R₀ is selected from the group consisting of optionally substituted alkyl or aryl, SRx, ORx and NRxRy wherein Rx and Ry are selected from H and organic residues and at least Rx is a substituent other than H.

R is preferably selected from a methyleneacyloxy group, a methylene carbonate group and a methylene carbamate group, and R″ is a leaving group. Preferred definitions of R when it is selected from a methyleneacyloxy group, a methylene carbonate group and a methylene carbamate group are given above. It has been found that the sulfurization agent according to the invention allows for particularly efficient sulfur transfer, in particular to form S-protected phosphorthioate internucleotide linkages in oligonucleotides. The sulfurizing agent according to the invention introduces a protected sulfur from which the protective group can be cleaved selectively and efficiently.

In the sulfurizing agent according to the invention, the leaving group R″ is generally an electrophilic group. Often, R″ is a group containing an electrophilic nitrogen atom bonded to the sulfur. The electrophilic nitrogen atom is suitably substituted with at least one electron-withdrawing group.

In a particular embodiment of the sulfurizing agent of the invention, the sulfurizing agent corresponds to formula (II)

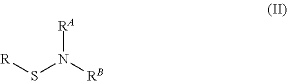

wherein R^{A} and R^{B} are equal or different from each other and at least one of R^{A} and R^{B} is selected from substituted sulfonyl or an acyl group, said R^{A} and R^{B} optionally forming together a cyclic substituent. Preferably, R denotes R¹—C(O)—O—CH₂—S, and in such instance, the formula (II) is as follows:

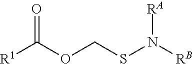

R¹ is preferably a C1-C20, optionally unsaturated or aromatic, hydrocarbon residue, preferably a linear or branched alkyl group or a cycloalkyl group. When at least one, preferably one, of R^{A} and R^{B} is substituted sulfonyl, it is generally selected from alkyl and aryl sulfonyl groups. When, preferably, at least one of R^{A} and R^{B} is an alkyl sulfonyl group, the alkyl substituent therein is preferably selected from lower alkyl or cycloalkyl (e.g., C1-C7) residues. Particular alkyl groups are selected from methyl, ethyl, n-propyl, isopropyl, n-butyl, sec-butyl, tert-Butyl, cyclopentyl and cyclohexyl. A methyl, ethyl or n-propyl group is preferred. A methyl group is more particularly preferred. When, at least one of R^{A} and R^{B} is an aryl sulfonyl group, the aryl substituent therein is, for example, an, optionally substituted, phenyl group. When at least one, preferably both, of R^{A} and R^{B} is an acyl group, it is generally selected from alkyl and aryl acyl groups. When, preferably, at least one of R^{A} and R^{B} is an alkyl acyl group, the alkyl substituent therein is preferably selected from lower alkyl or cycloalkyl (C1-C7) residues. Particular alkyl groups are selected from methyl, ethyl, n-propyl, isopropyl, n-butyl, sec-butyl, tert butyl, cyclopentyl and cyclohexyl. A methyl, ethyl or n-propyl group is preferred. A methyl group is more particularly preferred. In a particularly preferred embodiment, R^{A} and R^{B} are acyl groups forming together a cyclic substituent, preferably a 4 to 7 membered ring.

Preferably, in the sulfurizing agent of the present invention, R″ is a sulfonamide group, more preferably, an N-substituted-N-alkylsulfonyl.

In a specific embodiment, the sulfurizing agent corresponds to formula (III)

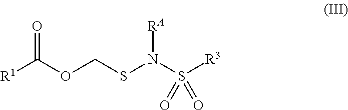

wherein R¹, R³ and R⁴ are independently a C1-C20, optionally unsaturated or aromatic, hydrocarbon residue, preferably a linear or branched alkyl group or a cycloalkyl group.

In a further specific embodiment of the sulfurizing agent according to the invention R″ is a dicarboxylamide. In a particular aspect of this embodiment the sulfurizing agent corresponds to formula (IV)

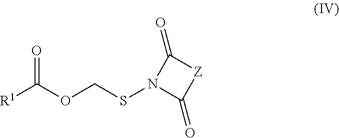

wherein Z is a group, chosen among the group of —CH₂—CH₂—, —CH═CH—, —CH₂—O—CH₂—,

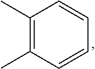

preferably Z is a

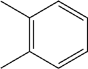

group.

A fourth particular object of the invention relates to a process for the synthesis of the sulfurizing agent according to the invention which comprises (a) reacting a sulfuryl halide, preferably sulfuryl chloride with a thioacetal of formula R—S—C(O)—R₂ wherein R is as described previously and R₂ is an organic residue, preferably selected from a C1-C20 optionally unsaturated or aromatic hydrocarbon residue to produce an intermediate product of formula R—S—W, wherein W is halogen preferably Cl and, (b) reacting said intermediate product with an N-sulfonyl compound or an N-acyl compound. In more specific embodiment of this invention, the thioacetal is of formula R₁—C(O)—O—CH₂—S—C(O)—R₂ wherein R₁ and R₂ are independently a C1-C20 optionally unsaturated or aromatic hydrocarbon residue and said thioacetal is reacted with sulfuryl chloride to produce an intermediate product of formula R₁—C(O)—O—CH₂—S—Cl, wherein R₁ is independently a C1-C20, optionally unsaturated or aromatic, hydrocarbon residue. In another specific embodiment of this invention, in step (b) the intermediate is reacted with an N-sulfonyl compound of formula R₃—S(O)₂—NH—R₄, wherein R₃ and R₄ are independently organic residues, preferably a C1-C20, optionally unsaturated or aromatic, hydrocarbon residue.

In an alternative, the process for the manufacture of the sulfurizing agent relates to the manufacture of dicarboxylamides. Here, the intermediate is reacted in step (b) with an N-acyl compound of formula

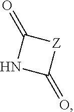

wherein Z, is an group chosen among the group of —CH₂—CH—, —CH═CH—, —CH₂—O—CH₂—,

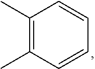

preferably Z is a group.

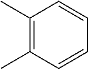

R1 is preferably selected from lower alkyl or cycloalkyl (C1-C7), phenyl including substituted phenyl and naphthyl groups, more preferably an ethyl group. R2, R3 and R4 are preferably selected from lower alkyl or cycloalkyl (C1-C7), phenyl including substituted phenyl and naphthyl groups, more preferably a methyl group.

In the process according to the invention for the synthesis of a sulfurizing agent, the reaction of step (a) is generally carried out in an aprotic polar organic solvent such as for example a halogenated hydrocarbon solvent, in particular a chlorinated hydrocarbon solvent such as methylene chloride.

In the process according to the invention for the synthesis of a sulfurizing agent, the reaction of step (a) is generally carried out at a temperature of from −80° C. to 30° C.

In the process according to the invention for the synthesis of a sulfurizing agent, the reaction of step (b) is generally carried out in an aprotic polar organic solvent such as for example a halogenated hydrocarbon solvent, in particular a chlorinated hydrocarbon solvent such as methylene chloride.

In the process according to the invention for the synthesis of a sulfurizing agent, the reaction of step (b) is generally carried out at a temperature of from −20° C. to 50° C., preferably from 0° C. to 30° C.

A fifth particular object of this invention concerns a method for manufacturing an oligonucleotide using the sulfurizing agent according to the invention. The method for synthesizing an oligonucleotide comprises using the sulfurizing agent for sulfurizing at least one phosphorus internucleotide linkage of a precursor of said oligonucleotide.

Generally, the method according to the invention comprises at least (a) a coupling step wherein a phosphorus internucleotide linkage is formed between two reactants selected from nucleotides and oligonucleotides and (b) a sulfurization step wherein the sulfurizing agent according to the invention is used to sulfurize said phosphorus internucleotide linkage. Steps (a) and (b) can be repeated after 3′ or 5′ deprotection of the sulfurized oligonucleotide. Preferably, the phosphorus internucleotide linkage is an H-phosphonate diester bond.

Step (a) of said manufacturing method preferably comprises forming the H-phosphonate diester bond by coupling an H-phosphonate monoester salt with a protected nucleoside or oligonucleotide having a free hydroxy group. The coupling is preferably carried out in solution phase.

Step (a) is preferably carried out in an aprotic polar organic solvent for example a halogenated solvent or nitrogen containing solvents, more particularly N-heterocyclic solvents or chlorinated hydrocarbon, even more particularly acetonitrile and pyridine and preferably pyridine. The reaction to form an H-phosphonate diester is preferably activated by a carboxylic acid halide, in particular pivaloyl chloride.

Step (a) is generally carried out at a temperature from −40° C. to 30° C., preferably from 0° C. to 20° C.

In step (a) the process according to the invention and in the particular embodiments thereof, the liquid reaction medium generally contains at least 10% by weight of H-phosphonate oligonucleotide relative to the total weight of the reaction medium. Preferably this content is at least 20% weight. The liquid reaction medium generally contains at most 50% by weight of H-phosphonate oligonucleotide relative to the total weight of the reaction medium.

The coupling product of step (a), in particular an H-phosphonate, may be isolated and subsequently sulfurized in step (b). It may also, preferably, be used without isolation in step (b). Sulfurization of formed diester can be carried by in-situ addition of the sulfurizing reagent, suitably dissolved in an appropriate solvent, or after pre-purifying formed diester from the reaction mixture.

Step (b) is preferably carried out in an aprotic polar organic solvent such as for example a solvent comprising a halogenated hydrocarbon solvent, in particular a chlorinated hydrocarbon solvent such as methylene chloride. In a particular aspect, step (b) is carried out in a solvent mixture comprising a halogenated hydrocarbon solvent and nitrogen containing solvents, more particularly N-heterocyclic solvents, preferably pyridine. A pyridine/methylene chloride mixture is more particularly preferred, in particular when the coupling product of step (a) is sulfurized without isolation.

Step (b) is generally carried out at a temperature of from −40° C. to 30° C., preferably from 0° C. to 20° C.

In step (b), the molar ratio of sulfurizing agent relative to the amount of internucleotide linkages to be sulfurized is generally at least 1, often from 1.5 to 4.0, preferably from 2.0 to 3.0.

In step (b) the intermediate H-phosphonate diester is preferably activated by an activator, in particular a base. Suitable bases include alkylamines, in particular tertiary alkylamines, diisopropylethylamine is preferred.

A sixth particular object of this invention concerns a method for manufacturing an oligonucleotide using the sulfurizing agent according to the invention and at least one sulfurizing agent of formula R″—S—R′ wherein R′ and R″ are as described above. Specifically, R′ is an organic residue other than the group R, preferably selected from a group consisting of an aryl group and a heteroaryl group which is bonded to the S-atom through an annular carbon atom, and R″ is a leaving group

It has been found that the sulfurization agents of formula R″—S—R and R″—S—R′ allow for particularly efficient sulfur transfer, in particular to form S-protected phosphorthioate internucleotide linkages in oligonucleotides. Said sulfurizing agents introduce a protected sulfur. The presence of different protective groups on the sulfur which have substantially different reactivity, allows for selective deprotection so as to be able to selectively create sulfurized or oxygenated internucleotide bonds. Consequently, the method of the invention allows the preparation of oligonucleotide sequences with a combination of both phosphodiester and phosphorothioate diester internucleotide linkages.

In a specific embodiment, the sulfurizing agent of formula R″—S—R′corresponds to formula (V)

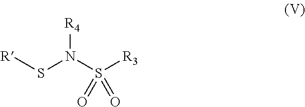

wherein
R′ is selected from a group consisting of an aryl group and a heteroaryl group which is bonded to the S-atom through an annular carbon atom; preferably R′ is an unsubstituted or substituted phenyl group, in particular a 4-halophenyl group or a 4-alkylphenyl group, for example a 4-(C1-C4-alkyl)phenyl group; R′ is especially a 4-chlorophenyl group; and R3 and R4 are independently a C1-C20, optionally unsaturated or aromatic, hydrocarbon residue, preferably a linear or branched alkyl group or a cycloalkyl group.

In another specific embodiment, the sulfurizing agent of formula R″—S—R′ corresponds to formula (VI)

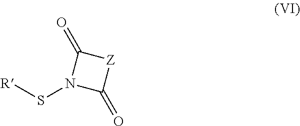

wherein
R′ is selected from a group consisting of an aryl group and a heteroaryl group which is bonded to the S-atom through an annular carbon atom; preferably R′ is an unsubstituted or substituted phenyl group, in particular a 4-halophenyl group or a 4-alkylphenyl group, for example a 4-(C₁-C₄-alkyl)phenyl group; R′ is especially a 4-chlorophenyl group; and Z is a group, chosen among the group of —CH₂—CH₂—, —CH═CH—, —CH₂—O—CH₂—,

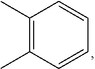

preferably Z is a

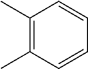

group.

The sulfurizing agent of formula (VI) can for example be prepared by a process which comprises (a) reacting a sulfuryl halide, preferably sulfuryl chloride with a thiol of formula R′—S—H wherein R′ is aromatic selected from the group consisting of aryl, alkylaryl, haloaryl, nitroaryl, alkoxyaryl to produce an intermediate product of formula R′—S—W, wherein R′ is described as above and W is halogen preferably Cl and, (b) reacting said intermediate product with an N-sulfonyl compound of formula R₁—NH—S(O)₂—R₂, wherein R₁ and R₂ are independently organic residues, preferably a C1-C20, optionally unsaturated or aromatic, hydrocarbon residue.

Generally, the method for the manufacture of oligonucleotides having at least one P—S—R internucleotide linkage and at least one P—S—R′ internucleotide linkage comprises at least

(a) a first coupling step wherein a first phosphorus internucleotide linkage is formed between two reactants selected from nucleotides and oligonucleotides and

(b) a sulfurization step wherein the sulfurizing agent according to the invention is used to sulfurize said first phosphorus internucleotide linkage and

(c) a second coupling step wherein a second phosphorus internucleotide linkage is formed between two reactants selected from nucleotides and oligonucleotides and

(d) a second sulfurization step wherein the sulfurizing agent of formula R″—S—R′ wherein R′ and R″ are as described above, is used to sulfurize said second phosphorus internucleotide linkage.

wherein steps (a) and (b) can be carried out before or after steps (c) and (d).

The method according to the invention suitably comprises more than 1, typically 2, 3, 4, 5, 7, 8, 9 or 10 sequences of steps (a) and (b). The method according to the invention suitably comprises more than 1, typically 2, 3, 4, 5, 7, 8, 9 or 10 sequences of steps (c) and (d). The order of sequences of steps (a) and (b) or (c) and (d) respectively is suitably determined on account of the desired pattern of respective phosphodiester and phosphorothioate diester internucleotide linkages. Typically, 3′ or 5′ deprotection of the sulfurized oligonucleotide is carried out after each sequence of step (a) and (b) or (c) and (d) respectively.

Preferably, the phosphorus internucleotide linkage is an H-phosphonate diester bond.

Step (a) of said manufacturing method preferably comprises forming the H-phosphonate diester bond by coupling an H-phosphonate monoester salt with a protected nucleoside or oligonucleotide having a free hydroxy group. The coupling is preferably carried out in solution phase.

The definitions and preferences described for step (a) and (b) above for the method for the manufacture of oligonucleotides having at least one P—S—R internucleotide linkage equally apply to the method for the manufacture of oligonucleotides in accordance with the invention having at least one P—S—R bond internucleotide linkage and at least one P—S—R′ internucleotide linkage.

In a seventh aspect, the invention, relates to a method for purifying an oligonucleotide in accordance with the invention having at least one P—S—R linkage as described herein before. In one embodiment of this aspect, the method comprises at least precipitating the oligonucleotide. In more specific embodiments, this method further comprise extraction of the oligonucleotide, in particular from solid material recovered from the precipitation step, with a solvent. Suitable solvents for extraction include a polar organic solvent

It has been found that the purification can be effectively accomplished by a combination of precipitation and extraction techniques of the protected oligonucleotide obtained according to the described method. The exact conditions of precipitation can be determined on account of given sequence and length of the oligonucleotide. The precipitation method generally comprises (a) dissolving the oligonucleotide in a polar organic solvent and (b) adding a non-polar organic solvent until the solution becomes turbid.

It has been found that the oligonucleotides according to the invention can generally be isolated and purified by precipitation.

The solvent used to dissolve the oligonucleotide in step (a) is preferably selected from halogenated hydrocarbons such as methylene chloride and chloroform, nitrogen containing solvents such as acetonitrile and pyridine, and carbonyl-containing solvents such as acetone.

Generally, in step (a), a solvent volume is used ranging from about 0.5 (n+1) mL to about 2.0 (n+1) mL. Preferably, about 1.0 (n+1) mL, where n is the millimoles number of phosphorothioate triester linkages.

The solution of the oligonucleotide is treated with a non-polar organic solvent preferably selected from hydrocarbons, for example alkane solvents such as hexane, ether solvent in particular MTBE, and their mixtures, such as, preferably hexane/MTBE mixtures until the solution becomes turbid. In another particular embodiment the turbid solution is subsequently treated with a precipitation aid.

In this case the precipitation aid is generally selected from inert porous solids preferably selected from Celite (a diatomaceous earth), charcoal, wood cellulose and chromatography stationary phases such as silica or alumina.

In this case, the precipitation aid is generally used in an amount ranging from about 0.25 (n+1) g to about 1.5 (n+1) g, preferably, about 0.75 (n+1) g, where n is the millimoles number of phosphorothioate triester linkages.

Preferably, after adding the precipitation aid the mixture is treated with a second fraction of a non-polar organic solvent as described here before. The volume of said fraction generally ranges from about 1(n+1) mL to about 4(n+1) mL, preferably, about 2.0 (n+1) mL, wherein n is the millimoles number of phosphorothioate triester linkages.

After precipitation, in particular when a precipitation aid is used, the obtained mixture is generally subjected to a solid/liquid separation operation such as, preferably, a filtration. The solid materials obtained in the precipitation step may be filtered off and washed.

The oligonucleotide is generally recovered from solid recovered from solid/liquid separation operation, in particular from precipitation aid by extraction with a polar organic solvent preferably selected from carbonyl-type solvents such as acetone, from nitrogen-containing solvents such as acetonitrile or a formamide type solvent, from polar ethers such as tetrahydrofurane, from halogenated hydrocarbons such as methylene chloride and chloroform or an aliphatic alcohol. The polar organic solvent is preferably selected from acetonitrile, tetrahydrofurane (THF), N,N-dimethylformamide (DMF), and an aliphatic alcohol.

The oligonucleotide obtained from the above precipitation treatment can be further purified by partitioning between an organic solvent, especially a polar organic solvent, and water. This step usually separates polar impurities, which dissolve in aqueous layer, from the product. In this embodiment, the oligonucleotide is suitably dissolved in a organic solvent, in particular a polar organic solvent in particular selected from acetonitrile, acetone, tetrahydrofurane (THF), N,N-dimethylformamide (DMF), and an aliphatic alcohol.

The volume of organic solvent used is generally ranging from 2.0 (n+1) mL to 8.0 (n+1) mL, preferably, about 4.0 (n+1) mL, where n is the millimoles number of the phosphorothioate triester linkage. The solution is treated with an aqueous medium, in particular water. The volume of aqueous medium used is generally from about 0.5 volume equivalent of the organic solvent to about 1.5 volume equivalent of the organic solvent, usually about 0.7 volume equivalent of the organic solvent.

In one preferred embodiment, the solvent comprises a mixture of polar organic solvent, preferably selected from acetonitrile, acetone, THF, DMF, with an aqueous medium, preferably water and wherein the volume ratio polar organic solvent/aqueous medium is preferably from about 0.5 to about 1.5, more preferably about 0.7. After treatment with the aqueous medium, an oligonucleotide-containing layer is generally separated and can be further processed, if appropriate, to obtain purified oligonucleotide.

Accordingly, a purified oligonucleotide which comprise at least one internucleotide linkage comprising a P—S—R bond and at least two nucleosides, wherein R corresponds to the formula (I)

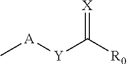

wherein A is a geminally substituted alkylene group, preferably CH₂, X and Y are independently selected from S and O, and R₀ is selected from the group consisting of optionally substituted carbon bonded organic residue, such as in particular optionally substituted alkyl or aryl, SRx, ORx and NRxRy wherein Rx and Ry are selected from H and organic residues and at least Rx is a substituent other than H, can be provided.

In an eight aspect, the invention, relates to a method for the manufacture of a purified oligonucleotide wherein the oligonucleotide in accordance with the invention having at least one P—S—R linkage and at least one P—S—R′ linkage as described herein before, is purified.

The definitions and preferences described for purifying an oligonucleotide in accordance with the invention having at least one P—S—R linkage equally apply for the method for purifying an oligonucleotide in accordance with the invention having at least one P—S—R internucleotide linkage and at least one P—S—R′ internucleotide linkage.

A ninth particular object of this invention concerns a method for producing an oligonucleotide from the oligonucleotides described above wherein the P—S—R group or groups and/or the P—S—R′ group or groups is/are cleaved. Especially, the ninth particular object of this invention concerns a method for producing a second oligonucleotide having at least one phosphorothioate group, which comprises (a) providing a first oligonucleotide according to the invention and (b) cleaving at least one R group, from said first oligonucleotide to produce said second oligonucleotide having at least one thiophosphate linkage. It has to be noted that the terms “thiophosphate linkage” and “phosphothioate” are used interchangeably. In some specific embodiments of the invention the R group is cleaved by reacting the first oligonucleotide in solution with a base chosen preferably from alkyl, cycloalkyl and aromatic amines, more preferably from primary, for example an alkyl primary amine wherein the alkyl group bears identical or different substituents selected preferably from C1 to C8 linear or branched alkyl, or secondary alkyl amines. Most preferably, the base is chosen from n-propyl and tert-butyl amine.

Preferably the base is a hindered primary amine.

In a particular embodiment, the cleavage according to the ninth aspect of the invention is carried out in the presence of a sterically hindered base and of an activator which is generally a N-heteroaromatic base. Preferably the activator is 1,2,4-triazole or selected from other triazole and tetrazole derivatives, and more preferably such activator is used with a sterically hindered base, in particular tert-butyl amine.

The deprotection of S-methylene-ester, -carbonate or -carbamate group can be accomplished for example in a treatment of protected nucleotide with a sterically hindered base such as e.g. t-butylamine. These bulky amines are particularly selective because they do not react with the nucleobases, particularly those protected at carbonyl oxygen. They allow in fact limiting or substantially avoiding possible side-reactions with the nucleobase moiety. In order to improve reactivity of sterically hindered amines with, for example, S-methylenepropanoate under standard conditions, it was found that an activator may suitably be added. Examples of activators which are suitable include N-heterocyclic bases such as e.g. diazole, triazole, and their derivatives. This embodiment allows particularly clean, fast and efficient deprotection reactions.

According to one embodiment, the first base is an alkyl primary amine wherein alkyl group bears identical or different substituents selected from C1 to C20 linear or branched alkyls.

According to another embodiment, the first base is an aryl primary amine wherein aryl group contains linear or branched alkyl or aryl groups at 2 and/or 6 positions.

In some embodiments of this invention the deprotection method involves using a substituted aniline as base wherein the aryl group of the aniline contains linear or branched alkyl or aryl substituents at 2 and/or 6 positions such as e.g. 2,6-dimethylaniline and 2,6-diethylaniline.

The deprotection according to the ninth aspect is preferably carried out in an aprotic polar organic solvent for example a solvent comprising nitrogen containing solvents, more particularly N-heterocyclic solvents, preferably pyridine.

The deprotection according to the ninth aspect is generally carried out at a temperature from −10° C. to 50° C., preferably from 0° C. to 30° C.

In the ninth aspect of the invention and in the particular embodiments thereof, the liquid reaction medium generally contains at least 20% by weight of first oligonucleotide relative to the total weight of the reaction medium. Preferably this content is at least 50% weight.

In the ninth aspect of the invention, the amount of base used is generally ranging from 5n mmol to 15n mmol, preferably about 10n mmol, where n is the millimoles number of the phosphorothioate triester linkage.

When an activator is used in the ninth aspect of the invention, the amount of activator used is generally ranging from 0.5n mmol to 3n mmol, preferably, 1.5n mmol, where n is the millimoles number of the phosphorothioate triester linkage.

A tenth particular object of this invention concerns a method for producing a fifth oligonucleotide having at least one phosphothioate linkage and at least one phosphodiester linkage, which comprises

(a) providing a third oligonucleotide in accordance with the invention having at least one P—S—R internucleotide linkage and at least one P—S—R′ internucleotide linkage and

(b) cleaving at least one R group, from said third oligonucleotide to produce a fourth oligonucleotide having at least one thiophosphate diester linkage and,

(c) subsequently cleaving at least one R′ group, from said fourth oligonucleotide to produce a fifth oligonucleotide having at least one phosphothioate linkage and at least one phosphodiester linkage.

The information concerning the cleavage of the R group from the first oligonucleotide equally applies for the cleavage of the R group from the third oligonucleotide. Thus:
Preferably, the R group, is cleaved by reacting the first oligonucleotide in solution with a base chosen preferably from alkyl, cycloalkyl and aromatic amines, more preferably from primary or secondary alkyl amines, most preferably from n-propyl and tert-butyl amine.
Preferably, the base is a hindered primary amine.
Preferably, the cleavage is carried out in the presence of a sterically hindered base and of an activator which is generally a N-heteroaromatic base.
Preferably, the sterically hindered base is t-butylamine.
Preferably, the activator is 1,2,4-triazole or other triazole and tetrazole derivatives.

Generally, the cleavage of the R group from the third oligonucleotide, can be carried out without or without substantial cleavage of the R′ group.

“without substantial cleavage” is understood to denote in particular a cleavage wherein less than 10% preferably less than 5% and more preferably less than 1% of the initially present R′ groups are cleaved.

Generally, the cleavage of the protecting R′ group on the phosphorothioate internucleotide linkage proceeds without cleavage of the formed internucleotide phosphate ester bond.

The cleavage of the protecting R′ group on the phosphorothioate internucleotide linkages can be carried out for example by oximate treatment, in particular with the conjugate base of an aldoxime. Suitable examples of conjugate bases of aldoximes are E-2-nitrobenzaldoxime, syn-pyridine-2-carboxaldoxime, E-4-nitrobenzaldoxime Said cleavage leads in general to phosphodiester internucleotide linkages.

Especially phenyl and substituted phenyl groups, in particular as described above, on the phosphorothioate internucleotide linkages can be removed by oximate treatment.

The following examples are intended to illustrate the invention without, however, limiting its scope

### EXAMPLES

In these examples and throughout this specification the abbreviations employed are defined as follows: CH₂Cl₂ is dichloromethane, KI is potassium iodide, Na₂S₂O₃ is sodium thiosulfate, DME is dimethoxyethane, DIPEA is diisopropylethylamine, NaCl is sodium chloride, MTBE is methyl tert-butyl ether, EtOAc is ethylacetate, HCl is hydrochloride acid, Na₂SO₄ is sodium sulfate, N₂ is dinitrogen, Br₂ is bromine, SO₂Cl₂ is thionyl chloride, NaHCO₃ is sodium bicarbonate, CDCl₃ is deuterated chloroform, THF is tetrahydrofurane, DMSO is dimethylsulfoxide, DMF is N,N-dimethylformamide.

Ap, Gp, Tp are the 2-deoxyribose nucleobases as previous described respectively connected to A, G and T nucleobases as previously described wherein A, G and T are protected as follows:

Ap is the 2-deoxyribose nucleobase wherein A is N-(purin-6-yl)benzamide, Gp is the 2-deoxyribose nucleobase wherein G is N-(6-(2,5-dichlorophenoxy)-purin-2-yl)isobutyramide and Tp is the nucleobase wherein T is 5-methyl-4-phenoxypyrimidin-2-one.

Ap(S), Gp(S) and Tp(S) are the corresponding 4′O—P-thiomethyl propionates of respectively Ap, Gp and Tp as previously described. Ap(H), Gp(H) and Tp(H) are the corresponding 4′O—P—H phosphonates of respectively Ap, Gp and Tp as previously described.

DMTr is the bis para-methoxy trityl protecting group, known to one skilled in the art, bonded to the 5-O′ of the corresponding oligonucleotide as previously described, when linked to it. Lev is the pentanl, 4-dione protecting group, known to one skilled in the art, bonded to the 3-O′ of the corresponding oligonucleotide as previously described, when linked to it.

The convention of abbreviations used in the description of invention is further illustrated by the following schemes.

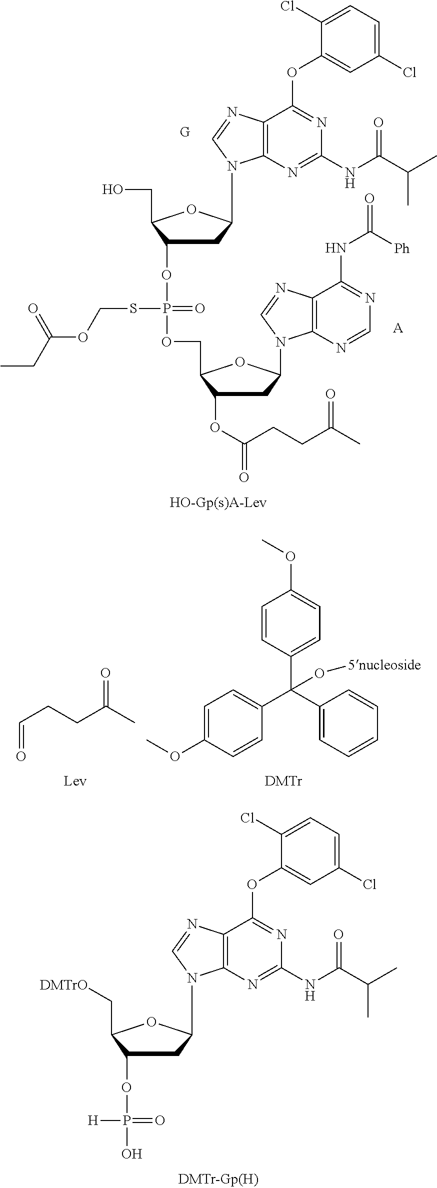

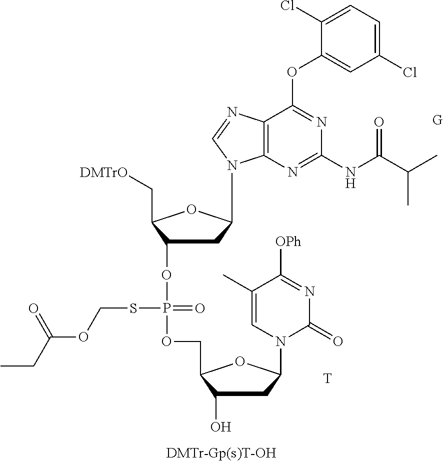

### Example 1

### Synthesis of bis-chloromethyl disulfide

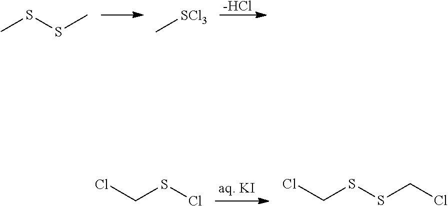

To a 1.0 L round bottom flask were added anhydrous CH₂Cl₂ (200 mL) and dimethyl disulfide (27.1 mL, 300 mmol). The mixture was stirred and cooled to −78° C. under a N₂ atmosphere, and Cl₂ gas was bubbled slowly through the stirred mixture. The addition of Cl₂ gas was stopped after the mixture turned into a yellow-green slurry. The cold bath was removed and the mixture was warmed spontaneously to room temperature. A red solution was formed as the evolution of HCl was going out of the solution. The mixture was first bubbled with N₂ for 15 min, and then rotary evaporated to remove volatile CH₂Cl₂. To the residue, fresh CH₂Cl₂ (300 mL) was added. The solution was stirred in an ice-water bath, an aqueous solution of KI (139.4 g, 840 mmol) in water (200 mL) was added slowly over 15 min. The cold bath was removed and the mixture was stirred at ambient temperature for 2 hours. The organic layer was separated and stirred in an ice-water bath; an aqueous saturated Na₂S₂O₃ solution was added slowly until the color of I₂ disappeared. The organic layer was separated, followed by washing with water (100 mL). The organic layer was dried over Na₂SO₄, followed by concentration to give product (42.2 g) as red oil. Yield: (87.3%). This crude was used for next step without further purification.

### Example 2

### Synthesis of bis(propionyloxymethyl)disulfide

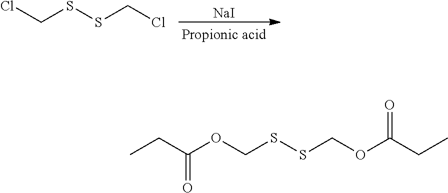

To a 1.0 L round bottom flask was added sodium iodide (1.50 g, 10.0 mmol), bis-chloromethyl disulfide (16.2 g, 100 mmol) and anhydrous DME (150 mL). After stirring at room temperature for 20 min, the mixture was cooled in an ice-water bath, and then DIPEA (42.0 mL, 240 mmol) was added, followed by propionic acid (16.4 mL, 220 mmol). The cold bath was removed and the mixture was stirred at room temperature for 18 hours. The bulk of the solvent was removed by rotary evaporation. Ethyl acetate (400 mL) was added to the residue, the mixture was then washed with water (150 mL×2), followed by brine (80 mL). The organic layer was concentrated and the residue was purified by silica gel chromatography to give the desired product (9.62 g) as an orange oil. Yield: (40.4%).

### Example 3

### Synthesis of N-methyl methanesulfonamide

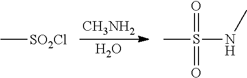

Methanesulfonyl chloride (38.7 mL, 500 mmol) was added dropwise over 15 min to stirred aqueous methylamine (40% in water, 152 mL, 1.75 mol) cooled in an ice-water bath. The internal temperature was kept between 20 and 24° C. during addition. After addition, the cooling bath was removed and the mixture was stirred at room temperature overnight. NaCl (40 g) was added and the mixture was stirred at room temperature for 30 min. The mixture was extracted with CH₂Cl₂ (150 mL, 100 mL×2). After drying over Na₂SO₄, the solvent was evaporated to give the desired product (48.7 g) as a colorless oil. Yield: 89.2%.

### Example 4

### Synthesis of N-methyl-N-propionyloxymethylsulfanyl methanesulfonamide

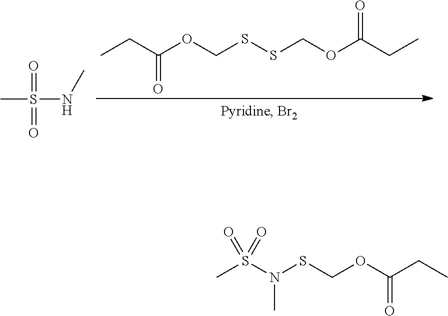

To a 100 mL dry round bottom flask was added N-methyl methanesulfonamide (1.09 g, 10.0 mmol), pyridine (1.66 g, 21.0 mmol), bis(propionyloxymethyl)disulfide (1.2 g, 5.0 mmol) and anhydrous CH₂Cl₂ (8 mL). The mixture was stirred under N₂ at room temperature and a solution of Br₂ (0.882 g, 5.52 mmol) in 4 mL of CH₂Cl₂ was added dropwise over 30 min. The resulting mixture was stirred at room temperature for 2 hours. MTBE (15 mL) was added and the resulting mixture was filtered. The solid was washed with a mixture of CH₂Cl₂ (5 mL) and MTBE (5 mL). The filtrate was concentrated and purified by silica gel chromatography to give the desired product (1.62 g) as a colorless oil. Yield: 71.3%.

### Example 5

### Synthesis of Chloromethyl Propionate

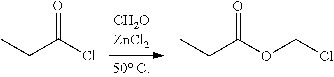

To a 500 mL dry round bottom flask was added paraformaldehyde (90.1 g, 3000.0 mmol), anhydrous zinc chloride (8.18 g, 60.0 mmol). The bottle was placed in an ice-water bath, and propionyl chloride (260.6 mL, 3000.0 mmol) was added slowly over 1 hour. After addition, the mixture was stirred at 50° C. under N₂ for 18 hours. The mixture was distilled to give the desired product 212.2 g as colorless oil. Yield: 58%.

### Example 6

### Synthesis of propionic acid acetylsulfanylmethyl ester

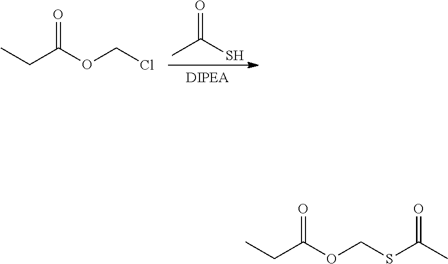

To a 2000 mL dry three-necked round bottom flask, equipped with a mechanical stirrer, a dropping funnel and a N₂ inlet, was added chloromethyl propionate (168.0 g, 1370.9 mmol), anhydrous CH₂Cl₂ (1000 mL), and diisopropylethylamine (194.9 g, 1508.0 mmol). To the stirred solution, cooled in an ice-water bath was added slowly thioacetic acid (98.0 mL, 1370.9 mmol) over 30 minutes. After addition, the mixture was stirred and warmed slowly to room temperature, and stirred at room temperature overnight. Most CH₂Cl₂ was rotary evaporated. To the mixture, a mixture of ethyl acetate (500 mL) and MTBE (500 mL) was added. The mixture was filtered, and the solid was washed with a mixture of ethyl acetate (100 mL) and MTBE (100 mL). The filtrate was concentrated and the residue was distilled to give the desired product (169.8 g) as yellow oil. Yield: 76.4%.

### Example 7

### Synthesis of N-methyl-N-propionyloxymethylsulfanyl methanesulfonamide

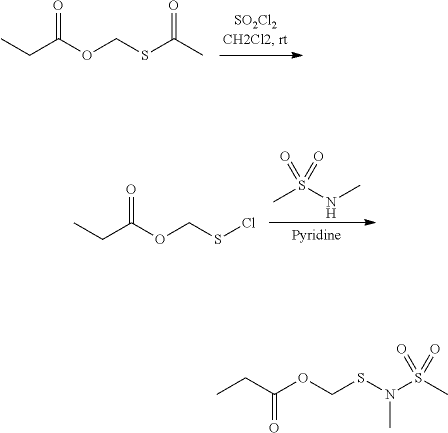

To a 1000 mL dry round bottom flask was added propionic acid acetylsulfanylmethyl ester (70.0 g, 431.5 mmol), anhydrous CH₂Cl₂ (600 mL). The solution was stirred in an ice water bath under N₂ and sulfuryl chloride (34.6 mL, 431.5 mmol) was added. After addition, the cold bath was removed and the mixture was stirred at room temperature for 1.5 hours. The mixture was rotary evaporated to remove all volatiles. The residue was dissolved in 80 mL of anhydrous CH₂Cl₂ to give solution A.

To another 1000 mL dry round bottom flask was added N-methyl methanesulfonamide (49.5 g, 453.1 mmol), molecular sieves (4 Å, activated, 5.0 g) and anhydrous CH₂Cl₂ (200 mL). The solution was stirred in an ice water bath under N₂, anhydrous pyridine (41.9 mL, 517.8 mmol) was added. After addition, the above solution A was added slowly over 15 minutes. The resulting mixture was then stirred at room temperature for 1.5 hours. Hexane (200 mL) was added slowly and the resulting mixture was stirred at room temperature for 10 minutes. The mixture was filtered, and the solid was washed with a mixture of ethyl acetate:hexane=1:1 (80 mL). The filtrate was concentrated and the residue was purified on a column using 800 g of silica and sequential elution starting with hexane (21) followed by ethyl acetate/hexane (1:9, 31), ethyl acetate/hexane (2:8, 41) and ethyl acetate/hexane (3:7, 21). The main impurity elutes at 20% of ethyl acetate and the product at 30% of ethyl acetate. Yield: 74.4%.

### Example 8

### Synthesis N-methanesulfonamide succinimide

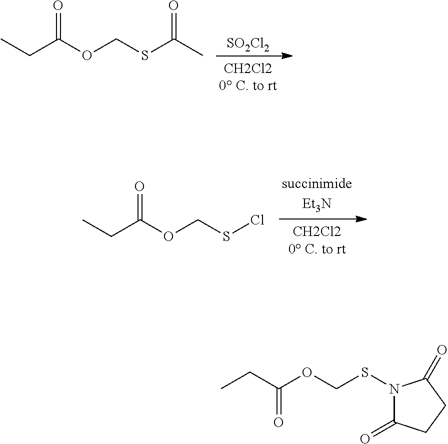

A solution of 9.803 g (60.43 mmol) of acetylsulfanylmethyl in 80 mL of anhydrous dichloromethane was placed in a 250 mL 3-neck round bottom flask equipped with a magnetic stirrer, thermocouple, nitrogen line and cooling ice bath. The flask's content was chilled to ˜0° C., and a total of 11.33 g (83.94 mmol, 1.38 eq.) of sulfuryl dichloride was slowly added to the solution with the rate to maintain temperature between 0 and 5° C. Cooling bath was removed, and reaction mixture was stirred at room temperature for additional 1.5 hour. The resulting yellow solution was concentrated on rotavap to yield 8.88 g of crude acetylmethylsulfenyl chloride as stench viscous yellow oil. This material was immediately used in the next step for the sulfenylation of succinimide.

A mixture of 6.12 g (61.76 mmol, 1.02 eq.) of succinimide and 8.17 g (80.73 mmol, 1.33 eq.) of triethylamine in 80 mL of anhydrous dichloromethane was placed in a 250 mL 3-neck round bottom flask equipped with a magnetic stirrer, thermocouple, nitrogen line and cooling ice bath. This mixture was cooled to 0° C., and a solution of crude acetylmethylsulfenyl chloride (8.88 g) in 20 mL of anhydrous dichloromethane was slowly added to the pre-chilled suspension of succinimide and triethylamine with the rate to maintain temperature between 0° C. and 5° C. After completion of addition, cooling bath was removed and resulting brown suspension was allowed to stir at room temperature for additional 2 hours. The reaction mixture was quenched with cold water (300 mL) and saturated NaHCO₃ (100 mL), and extracted with dichloromethane (3×80 mL). Organic phases were combined, dried over Na₂SO₄ and concentrated on rotavap to yield 13 g of dark viscous oil. This material was purify on silica gel column (120 g) using EtOAc-hexane mixture as eluent with 0 to 30% of ethylacetate to give 4.12 g of sulfenamide N-methanesulfonamide succinimide as white solid, R_{f}=0.16 in 30% EtOAc-hexane. Yield: 32%.

### Example 9

### Synthesis of Ethyl Acetylsulfanylmethyl Carbonate

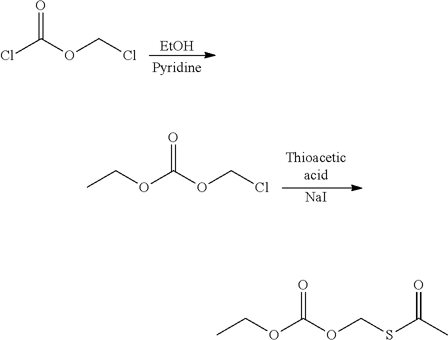

To a 100 mL dry round bottom flask is added chloromethyl chloroformate (12.9 g, 100.0 mmol), anhydrous acetonitrile (300 mL). The solution is stirred in an ice-water bath, and a mixture of anhydrous ethanol (4.6 g, 100.0 mmol) and anhydrous pyridine (23.7 g, 300 mmol) is added slowly over 20 min. After addition, the mixture is stirred at room temperature for 1 hour. Sodium iodide (1.50 g, 10.0 mmol) is added into the reaction mixture. The mixture is stirred in an ice-water bath, and thioacetic acid (7.6 g, 100 mmol) is added over 5 min. After addition, the cold bath is removed and the mixture is stirred at room temperature overnight. Hexane (600 mL) is added into the reaction mixture and is filtered. The filtrate is concentrated and distilled to give the desired product.

### Example 10

### Synthesis of N-methyl-N-ethoxycarbonyloxymethylsulfanyl methanesulfonamide

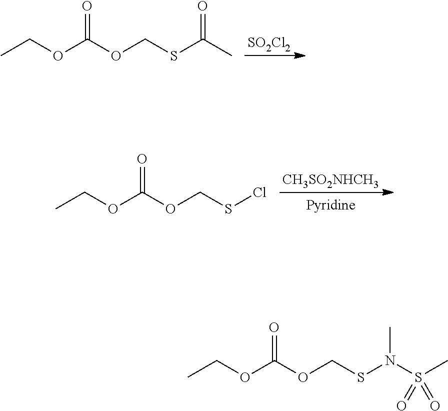

To a 500 mL dry round bottom flask is added ethyl acetylsulfanylmethyl carbonate (8.9 g, 50.0 mmol), anhydrous CH₂Cl₂ (200 mL). The solution is stirred in an ice water bath under N₂ and sulfuryl chloride (4.0 mL, 50.0 mmol) is added. After addition, the cold bath is removed and the mixture is stirred at room temperature for 1.5 hours. The mixture is rotary evaporated to remove all volatiles. The residue is dissolved in 50 mL of anhydrous CH₂Cl₂ to give solution A.

To another 500 mL dry round bottom flask is added N-methyl methanesulfonamide (6.0 g, 55.0 mmol), molecular sieves (4 Å, activated, 3.0 g) and anhydrous CH₂Cl₂ (150 mL). The mixture is stirred in an ice water bath under N₂, anhydrous pyridine (5.3 mL, 65.0 mmol) is added. After addition, the above solution A is added slowly over 10 minutes. The resulting mixture is then stirred at room temperature for 1.5 hours. Hexane (200 mL) is added slowly and the resulting mixture is stirred at room temperature for 10 minutes. The mixture is filtered, and the solid is washed with a mixture of ethyl acetate:hexane=1:1 (60 mL). The filtrate is concentrated and the residue is purified by a silica gel column to give the desired product.

### Example 11

### Synthesis of Acetylsulfanylmethyl Dimethylcarbamate

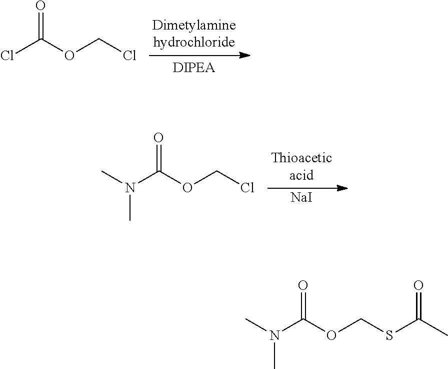

To a 100 mL dry round bottom flask is added chloromethyl chloroformate (12.9 g, 100.0 mmol), dimethylamine hydrochloride (8.15 g, 100 mmol) and anhydrous acetronitrile (300 mL). The mixture is stirred in an ice-water bath, and N,N-diisopropylethyl amine (43.5 mL, 250 mmol) is added slowly over 30 min. After addition, the mixture is stirred at room temperature for 1 hour. Sodium iodide (1.50 g, 10.0 mmol) is added into the reaction mixture. The mixture is stirred in an ice-water bath, and thioacetic acid (7.6 g, 100 mmol) is added over 5 min. After addition, the cold bath is removed and the mixture is stirred at room temperature overnight. Hexane (300 mL) is added into the reaction mixture and is filtered. The filtrate is concentrated and distilled to give the desired product.

### Example 12

### Synthesis of N-methyl-N-dimethylcarbamoyloxymethylsulfanyl methanesulfonamide

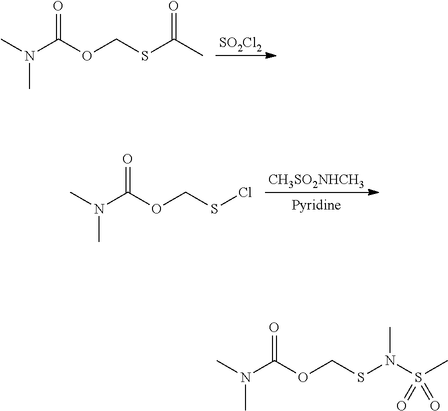

To a 500 mL dry round bottom flask is added ethyl acetylsulfanylmethyl dimethylcarbamate (8.9 g, 50.0 mmol), anhydrous CH₂Cl₂ (200 mL). The solution is stirred in an ice water bath under N₂ and sulfuryl chloride (4.0 mL, 50.0 mmol) is added. After addition, the cold bath is removed and the mixture is stirred at room temperature for 1.5 hours. The mixture is rotary evaporated to remove all volatiles. The residue is dissolved in 50 mL of anhydrous CH₂Cl₂ to give solution A.

To another 500 mL dry round bottom flask is added N-methyl methanesulfonamide (6.0 g, 55.0 mmol), molecular sieves (4 Å, activated, 3.0 g) and anhydrous CH₂Cl₂ (150 mL). The mixture is stirred in an ice water bath under N₂, anhydrous pyridine (5.3 mL, 65.0 mmol) is added. After addition, the above solution A is added slowly over 10 minutes. The resulting mixture is then stirred at room temperature for 1.5 hours. Hexane (100 mL) is added slowly and the resulting mixture is stirred at room temperature for 10 minutes. The mixture is filtered, and the solid is washed with a mixture of ethyl acetate:hexane=1:1 (60 mL). The filtrate is concentrated and the residue is purified by a silica gel column to give the desired product.

### Example 13

### Synthesis of Fully Protected Dinucleotide Phosphorothioate with Isolation of Intermediate H-Phosphonate

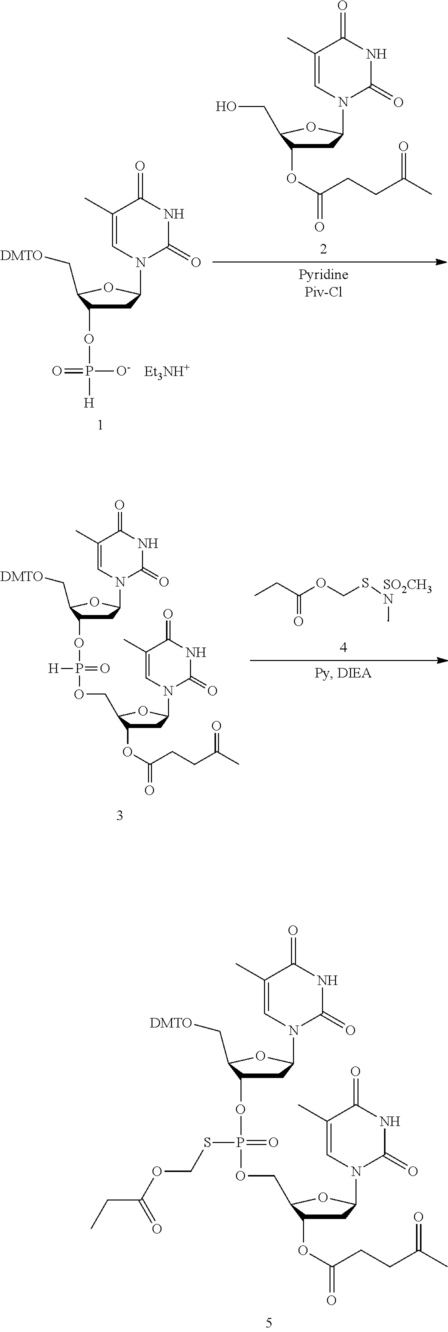

Example 13-1. A mixture of 1.86 g (2.62 mmol, 1.15 eq.) of H-phosphonate 1 and 0.78 g (2.28 mmol) of 3′-protected deoxy-thymidine 2 was co-evaporated with anhydrous pyridine (3×25 mL). The oily residue was dissolved in 10 mL of anhydrous pyridine and cooled to ˜0° C. under argon atmosphere. A total of 0.56 g (4.66 mmol, 2 eq.) of pivaloyl chloride was added dropwise via syringe, and the resulting mixture was allowed to warm up to ambient temperature. The reaction stirred for an additional 15 min, and then was quenched with 50 g of ice and 100 mL of diluted saturated NaHCO₃ (80 mL water and 20 mL of sodium bicarbonate). The organic material was extracted with dichloromethane (2×80 mL) and the extract was washed with a mixture of cold water (70 mL), saturated sodium bicarbonate (30 mL) and brine (10 mL). The organic layer was dried over anhydrous sodium sulfate and concentrated on rotavap to give 6.74 g of crude 3 as clear oil. ³¹P (162 MHz, CDCl₃, δ): 8.58 (s) and 7.06 (s).

A solution of crude H-phosphonate 3 (6.74 g) in 20 mL of anhydrous pyridine was cooled to ˜0° C. under argon atmosphere. A total of 1.21 g (5.43 mmol, 2.3 eq.) of reagent 4 was added drop wise to the reaction, and after 5 min 0.502 g (3.88 mmol, 1.7 eq.) of diisopropylethyl amine was also added to the flask. The reaction was allowed to warm up to ambient temperature, and after an additional stirring for 1 hours it was quenched with diluted cold solution of sodium bicarbonate (100 mL). Organic products were extracted with dichloromethane (2×80 mL), washed with water (100 mL) and dried over sodium sulfate. The organic phase was concentrated on rotavap to give 5.02 g of crude 5 (˜90% pure by HPLC) as yellow oil. ³¹P (162 MHz, CDCl₃, δ): 26.77 (s) and 26.67 (s).

Example 13-2. By analogy with the method A, an intermediate H-phosphonate 3 was obtained by reaction of H-phosphonate 1 (1.49 g, 2.1 mmol, 1.08 eq.) with 3′-protected deoxythymidine 2 (0.66 g, 1.94 mmol) in the presence of 0.49 g (4.06 mmol, 2 eq.) of pivaloyl chloride in 20 mL of anhydrous pyridine. After quenching of the reaction mixture with cold water/aq. NaHCO₃/brine, the intermediate 3 was isolated by extraction with dichloromethane (3×30 mL). The organic extract was washed with water (50 mL), aq. NaHCO₃ (20 mL) and brine (10 mL). After drying with Na₂SO₄ (for ˜1 min), the organic phase was concentrated to ˜¼ of the original volume, cooled to 0° C., and S-transfer reagent 4 (0.96 g, 4.3 mmol, 2.2 eq.) was added to 3 following by addition of diisopropylethylamine (0.45 g, 3.48 mmol, 1.8 eq.). After stirring at room temperature for an additional 1 hour, the reaction was quenched as described in Method A. Concentration of the organic phase on a rotavap yielded 3.55 g of crude 5 as clear yellow oil with HPLC purity of 91%.

### Example 14

### Preparation of DMTr-Ap(s)T-Lev

A mixture of triethylammonium 6-N-(bezoyl)-5′-O-(4,4′-dimethoxytrityl)-2′-deoxyadenosine-3′H-phosphonate (4.94 g, 6.0 mmol) and 3′-O-levulinylthymidine (1.70 g, 5.0 mmol) was rendered anhydrous by evaporation with pyridine, diluted with anhydrous pyridine (12.5 mL) and stirred under N₂ at 0° C. Subsequently, pivaloyl chloride (1.24 mL, 10.0 mmol) was added slowly over 2 min. The reaction mixture was stirred at 0° C. for 5 min and partitioned between methylene chloride (100 mL) and 1.25 N sodium acetate—acetic acid buffer (2×100 mL). The buffer was made by mixing 190 mL of 1.25 N aqueous sodium acetate solution with 10 mL of 1.25 N aqueous acetic acid solution. The organic layer was dried (Na₂SO₄) and concentrated. The residue was co-evaporated with 50 mL of toluene, dissolved in anhydrous methylene chloride (25 mL), and treated under N₂ at 0° C. with a solution of N-propionyloxymethylthio-N-methyl methanesulfonamide (2.05 g, 9.0 mmol) in anhydrous methylene chloride (1.0 mL), followed by the addition of N,N-diisopropylethylamine (0.87 mL, 5.0 mmol). After stirring at ambient temperature for 30 min, solution A (MTBE: Hexane=1:2, 37.5 mL) was added over 10 min, followed by Celite (7.5 g). The mixture was stirred and the additional portion of A (37.5 mL) was added over 30 min. The stirring was continued for further 30 min, and the mixture was filtered. The solid was washed with a mixture of solvent made of the solution A and CH₂Cl₂ in the ratio of 5:1 (60 mL). The solid then was extracted with methylene chloride (4×40 mL). The methylene chloride filtrate was concentrated. The residue was dissolved in acetonitrile (20 mL) and stirred in an ice-water bath, and cold water (14 mL) was added over 20 min. The bottom layer was partitioned between methylene chloride (100 mL) and aqueous (1:1) brine solution (60 mL). The organic layer was dried (Na₂SO₄) and concentrated to give product (5.7 g) as white solid. Yield: 97%. ³¹P NMR (CDCl₃, 121.5 MHz): δ=26.7, 26.3.

### Example 15

### Preparation of DMTr-Gp(H)

To phosphorous acid (78.7 g, 960.0 mmol) which was made anhydrous by evaporation with pyridine (500 mL), 2′-deoxy-6-O-(2,5-dichlorophenyl)-5′-O-(4,4′-dimethoxytrityl)-2-N-isobutyrylguanosine (62.8 g, 80.0 mmol) was added and the mixture was again rendered anhydrous by evaporation with pyridine. The residue was treated with anhydrous pyridine (480 mL) and pivaloyl chloride (64.0 mL, 520.0 mmol) which was added over 30 min at 10° C. The reaction mixture was stirred for 6 hours at room temperature and concentrated. The residue was dissolved in 800 mL of methylene chloride and washed sequentially with cold water (800 mL), triethylammonium hydrogen carbonate (2.0 N, 400 mL×2). The organic layer was dried (anhydrous Na₂SO₄) and concentrated. The residue was dissolved in 80 mL of methylene chloride, and a solution A (MTBE:Hexane=1:2, 360 mL) was added over 20 min under stirring followed by Celite (80 g). Subsequently, additional solution A (360 mL) was added slowly over 30 min. The mixture was filtered and the solid was washed with MTBE (300 mL). The solid was then extracted with methylene chloride (200 mL×4). The methylene chloride filtrate was concentrated to give product (70.4 g) as white foam. Yield: 93%. ³¹P NMR (CDCl₃, 121.5 MHz): δ=2.71.

### Example 16

### Preparation of DMTr-Gp(s)T-OH

A mixture of triethylammonium 2′-deoxy-6-O-(2,5-dichlorophenyl)-5′-O-(4,4′-dimethoxytrityl)-2-N-isobutyrylguanosine-3′H-phosphonate (54.9 g, 58.0 mmol) and 3′-O-levulinyl-4-O-phenylthymidine (20.1 g, 48.3 mmol) was rendered anhydrous by evaporation with pyridine and diluted with anhydrous pyridine (121 mL). This solution was treated under N₂ at 0° C. with pivaloyl chloride (11.8 mL, 96.6 mmol) over 5 min. After stirring for additional 5 min, a solution of N-propionyloxymethylthio-N-methyl methanesulfonamide (22.0 g, 96.6 mmol) in anhydrous methylene chloride (20 mL) was added, followed by N N-diisopropylethylamine (8.4 mL, 48.3 mmol). The reaction mixture was allowed to stir at ambient temperature for 30 min and was, diluted with 600 mL of methylene chloride. The organic layer was washed sequentially with cold water (600 mL), and saturated sodium bicarbonate (500 mL×2), dried (anhydrous Na₂SO₄) and concentrated. The residue was dissolved in 97 mL of methylene chloride, and a solution A (MTBE:Hexane=1:2, 194 mL) was added over 20 min under stirring followed by celite (73 g) and the additional portion of the solution A (194 mL) which was added over 30 min. After stirring for additional 30 min, the mixture was filtered. The solid was washed with MTBE:Hexane=4:1 (200 mL and was extracted with methylene chloride (150 mL×4). The methylene chloride filtrate was concentrated to give product (68.1 g) as yellow foam ³¹P NMR (CDCl₃, 121.5 MHz): δ=26.9, 26.2. This product was used for next step without further purification. To a stirred solution of the above product (64 g) in 117 mL of methylene chloride at 0° C., a cold mixture of pyridine: acetic acid: hydrazine monohydrate=37.5 mL: 25.0 mL: 2.5 mL (51.6 mmol) was added. After stirring at 0° C. for 1 hour, the reaction mixture was diluted with methylene chloride (200 mL) and was washed with cold water (500 mL). The aqueous layer was back extracted with methylene chloride (100 mL). The combined methylene chloride extracts were dried (anhydrous Na₂SO₄) and concentrated. The residue was dissolved in 235 mL of acetonitrile, stirred in an ice-water bath, and treated with cold water (188 mL) which was added gradually over 30 min. The bottom organic layer was separated, diluted with 200 mL of methylene chloride and dried (anhydrous Na₂SO₄). After concentration, the residue was dissolved in 94 mL of methylene chloride and treated sequentially under stirring by solution A (MTBE: Hexane=1:2, 94 mL) added over 20 min, Celite (70 g) and again by the solution A (94 mL) added over 30 min. The mixture was filtered, the solid was washed with MTBE: Hexane=4:1 (300 mL) and extracted with methylene chloride (150 mL×4). The methylene chloride filtrate was concentrated to give the product (52 g) as yellow foam. Yield: 87%. This product was used for next step directly. ³¹P NMR (CDCl₃, 121.5 MHz): δ=28.1, 25.3.

### Example 17

### Preparation of HO-Gp(s)A-Lev

A mixture of triethylammonium 2′-deoxy-6-O-(2,5-dichlorophenyl)-5′-O-(4,4′-dimethoxytrityl)-2-N-isobutyrylguanosine-3′H-phosphonate (45.4 g, 48.0 mmol) and 2′-deoxy-3′-O-levulinyl-6-N-benzoyladenosine (18.1 g, 40.0 mmol) was rendered anhydrous by evaporation with pyridine. The residue was diluted with anhydrous pyridine (100 mL), and the resulting solution was treated under N₂ at 0° C. with pivaloyl chloride (9.9 mL, 80.0 mmol) over 10 min. The reaction mixture was stirred at 0° C. for additional 5 min and treated with a solution of N-propionyloxymethylthio-N-methyl methanesulfonamide (18.2 g, 80.0 mmol) in anhydrous methylene chloride (10 mL), followed by N N-diisopropylethylamine (7.0 mL, 40.0 mmol). After stirring at ambient temperature for 30 min, the reaction mixture was diluted with 600 mL of methylene chloride and washed sequentially with cold water (600 mL), and saturated sodium bicarbonate (300 mL×2). The organic layer was dried (anhydrous Na₂SO₄) and concentrated. The residue was dissolved in 80 mL of methylene chloride, and treated sequentially with a solution A (MTBE: Hexane=1:2, 160 mL) over 20 min, followed by Celite (60 g) and additional portion of solution A (160 mL) over 30 min. After stirring for 30 min, the mixture was filtered. The solid was washed with MTBE: Hexane=4:1 (200 mL) and extracted with methylene chloride (150 mL×4). The methylene chloride filtrate was concentrated. The residue was dissolved in 160 mL of acetonitrile, stirred in an ice-water bath, and treated with cold water (112 mL) over 30 min. The bottom organic layer was separated and partitioned between methylene chloride (320 mL) and water (320 mL). The organic layer was dried (anhydrous Na₂SO₄) and concentrated to give product (64.1 g) as yellow foam. This product was used for next step without further purification. ³¹P NMR (CDCl₃, 121.5 MHz): 8=26.2, 26.0.

To a stirring solution of the above product (64.0 g) in 120 mL of methylene chloride at 0° C., pyrrole (13.9 mL, 200.0 mmol) was added followed by dichloroacetic acid (16.5 mL, 200.0 mmol) addition over 20 min. After stirring at 0° C. for 1 hour, the reaction mixture was quenched with saturated sodium bicarbonate (200 mL). The aqueous layer was extracted with methylene chloride (60 mL×2) and the combined methylene chloride extracts were dried (anhydrous Na₂SO₄) and concentrated. The residue was dissolved in 80 mL of methylene chloride, and treated sequentially with a solution A (MTBE: Hexane=1:1, 100 mL) over 15 min, Celite (60 g) and an additional portion of solution A (100 mL) over 30 min. The mixture was filtered, the solid was washed with MTBE (150 mL×2) and extracted with methylene chloride (200 mL×4). The methylene chloride filtrate was concentrated, dissolved in 160 mL of acetonitrile, stirred in an ice-water bath, and treated with cold water (144 mL) over 20 min. The bottom organic layer was separated and partitioned between methylene chloride (320 mL) and aqueous (1:1) brine solution (320 mL). The organic layer was dried (anhydrous Na₂SO₄) and concentrated to give product (40.5 g) as off-white solid. Yield: 92%. ³¹P NMR (CDCl₃, 121.5 MHz): 6=26.4, 26.2.

### Example 18

### Preparation of DMTr-Gp(s)Tp(H)

Phosphorous acid (29.8 g, 364.0 mmol) was rendered anhydrous by evaporation with pyridine (182 mL). To the residue, DMTr-Gp(s)T-OH (33.0 g, 26.0 mmol) was added and the mixture was again rendered anhydrous by evaporation with pyridine. The mixture was diluted with anhydrous pyridine (130 mL) and treated with pivaloyl chloride (24.0 mL, 195.0 mmol) which was added over 30 min at 10° C. The mixture was stirred for 16 hours at room temperature, concentrated, and the residue was dissolved in 400 mL of methylene chloride. The solution was washed sequentially with cold water (400 mL) and triethylammonium hydrogen carbonate (2.0 N, 200 mL×3). The organic layer was dried (anhydrous Na₂SO₄) and concentrated. After co-evaporating with 150 mL of toluene, the residue was dissolved in 52 mL of methylene chloride, and treated sequentially with a solution A (MTBE: Hexane=1:1, 78 mL) over 20 min, Celite (39 g) and an additional portion of the solution A (78 mL) over 30 min. The mixture was filtered and the solid was washed with a solvent mixture made from the solution A and CH₂Cl₂ in the ratio of 5:1 (180 mL). The solid was extracted with methylene chloride (150 mL×4), the filtrate was concentrated to give product (33.1 g) as off-white foam. Yield: 89%. ³¹P NMR (CDCl₃, 121.5 MHz): δ=26.9, 25.4, 3.0, 2.9.

### Example 19

### Preparation of DMTr-Gp(s)Tp(s)Gp(s)A-OH

A mixture of triethylammonium salt of DMTr-Gp(s)Tp(H) (28.6 g, 20.0 mmol) and HO-Gp(s)A-Lev (16.9 g, 15.4 mmol) was rendered anhydrous by evaporation with pyridine. The residue was diluted with anhydrous pyridine (62.0 mL) and treated under N₂ at 0° C. with pivaloyl chloride (4.8 mL, 38.5 mmol) over 5 min. After stirring at 0° C. for additional 10 min, a solution of N-propionyloxymethylthio-N-methyl methanesulfonamide (7.0 g, 30.8 mmol) in anhydrous methylene chloride (10 mL) was added, followed by N,N-diisopropylethylamine (2.7 mL, 15.4 mmol). After stirring at ambient temperature for 1 hour, the reaction mixture was diluted with 450 mL of methylene chloride and was washed sequentially with cold water (450 mL) and saturated sodium bicarbonate (300 mL×2). The organic layer was dried (anhydrous Na₂SO₄) and concentrated. After co-evaporation with 100 mL of toluene, the residue was dissolved in 62 mL of methylene chloride, and treated sequentially with a solution A (MTBE: Hexane=1:1, 124 mL) over 20 min, followed by Celite (46.2 g) and an additional portion of the solution A (124 mL) over 30 min. After stirring for 30 min, the mixture was filtered, the solid was washed with solution A: CH₂Cl₂=6:1 (140 mL). The solid then was washed with methylene chloride (150 mL×4). The methylene chloride filtrate was concentrated. The residue was dissolved in 123 mL of acetonitrile and treated with cold water (86 mL) over 20 min while stirring in an ice-bath. The bottom organic layer was separated and partitioned between methylene chloride (300 mL) and aqueous (1:1) brine solution (200 mL). The organic layer was dried (anhydrous Na₂SO₄) and concentrated to give product DMTr-Gp(s)Tp(s)Gp(s)A-Lev (43.4 g) as yellow foam. This product was used for next step without further purification. ³¹P NMR (CDCl₃, 121.5 MHz): 6=27.9-25.8 (m).

To a stirring solution of DMTr-Gp(s)Tp(s)Gp(s)A-Lev (38.0 g) in 38.0 mL of methylene chloride at 0° C., a cold mixture of pyridine:acetic acid:hydrazine monohydrate=14.3 mL:9.5 mL:0.95 mL (19.5 mmol) was added. After stirring at 0° C. for 40 min, the reaction mixture was diluted with methylene chloride (450 mL) and washed with cold water (300 mL×2) and brine (150 mL). The methylene chloride layer was dried (Na₂SO₄) and concentrated. After co-evaporated with 100 mL of toluene, the residue was dissolved in 60 mL of methylene chloride, and solution A (MTBE: Hexane=1:1, 90 mL) was added over 20 min under stirring followed by celite (45 g). The mixture was stirred and more solution A (90 mL) was added over 30 min. The mixture was filtered and the solid was washed with a solvent mixture made from the solution A and CH₂Cl₂ in the ratio of 5:1 (150 mL). The solid was extracted with methylene chloride (150 mL×4), the extract was concentrated and the residue was purified with a short silica column using a gradient of acetonitrile 0-80% in ethyl acetate. The product fractions were concentrated to give the product (26.9 g) as yellow foam. Yield: 82%. ³¹P NMR (CDCl₃, 121.5 MHz): δ=27.6-26.0 (m).

### Example 20

### Preparation of HO-Gp(s)Tp(s)Gp(s)A-Lev

To a stirred solution of DMTr-Gp(s)Tp(s)Gp(s)A-Lev (7.2 g, 2.84 mmol) in 14 mL of methylene chloride at 0° C., pyrrole (2.0 mL, 28.4 mmol) was added followed by dichloroacetic acid (2.34 mL, 28.4 mmol) over 3 min. After stirring at 0° C. for 30 min, the reaction mixture was diluted with 14 mL of methylene chloride, followed by slow addition of saturated sodium bicarbonate (50 mL). The aqueous layer was extracted with methylene chloride (40 mL) and combined methylene chloride extracts were dried (anhydrous Na₂SO₄) and concentrated. The residue was dissolved in 30 mL of methylene chloride, and treated sequentially with a solution A (MTBE: Hexane=2:1, 45.0 mL), Celite (9.0 g), and the solution A (45.0 mL) again over 30 min. The mixture was filtered, the solid was washed with MTBE (40 mL×2) and extracted with methylene chloride (50 mL×4). The methylene chloride extract was concentrated, the residue was dissolved in 28.4 mL of acetonitrile and treated while stirring in an ice-bath with cold water (28.4 mL) over 20 min. The organic layer was diluted with methylene chloride (80 mL) dried (anhydrous Na₂SO₄) and concentrated to give product (5.7 g) as off-white solid. Yield: 95%. ³¹P NMR (CDCl₃, 121.5 MHz): δ=27.4-26.2 (m).

### Example 21

### Preparation of DMTr-Gp(s)Tp(s)Gp(s)Ap(H)

Phosphorous acid (4.7 g, 57.3 mmol) was evaporated with pyridine (29 mL) and mixed with DMTr-Gp(s)Tp(s)Gp(s)A-OH (8.7 g, 3.58 mmol). The mixture was rendered anhydrous by was anhydrous by evaporation of added pyridine and diluted with anhydrous pyridine (29.0 mL). To the stirred mixture, pivaloyl chloride (3.75 mL, 30.4 mmol) was added over 5 min at 10° C. The mixture was stirred for 6 hours at room temperature and concentrated. The residue was dissolved in 200 mL of methylene chloride and washed sequentially with cold water (100 mL), and triethylammonium hydrogen carbonate (2.0 N, 100 mL×3). The organic layer was dried (anhydrous Na₂SO₄) and concentrated to give the product (9.15 g) as off-white foam. Yield: 98%. ³¹P NMR (CDCl₃, 121.5 MHz): δ=27.5-25.8 (m), 2.9, 2.8.

### Example 22

### Preparation of HO-Gp(s)Tp(s)Gp(s)Ap(s)Gp(s)Tp(s)Gp(s)A-Lev

A mixture of triethylammonium salt of DMTr-Gp(s)Tp(s)Gp(s)Ap(H) (10.1 g, 3.9 mmol) and HO-Gp(s)Tp(s)Gp(s)A-Lev (6.7 g, 3.0 mmol) was rendered anhydrous by evaporation with pyridine and diluted with anhydrous pyridine (15.0 mL). Pivaloyl chloride (1.1 mL, 9.0 mmol) was added slowly over 2 min to this mixture with stirring at 0° C. The mixture was stirred at ambient temperature for 30 min, cooled again at 0° C., and a solution of N-propionyloxymethylthio-N-methyl methanesulfonamide (1.70 g, 7.5 mmol) in anhydrous methylene chloride (2.0 mL) was added, followed by N N-diisopropylethylamine (0.78 mL, 4.5 mmol). The reaction mixture was stirred at ambient temperature for 1 hour, and diluted with 300 mL of methylene chloride. The organic layer was washed sequentially with cold water (300 mL) and saturated sodium bicarbonate (200 mL), dried (anhydrous Na₂SO₄) and concentrated. The residue was dissolved in methylene chloride (24 mL), and treated sequentially with a solution A (MTBE: Hexane=1:1, 48 mL) over 10 min, Celite (18 g), and an additional portion of the solution A (48 mL) over 30 min. After stirring for 30 min, the mixture was filtered, the solid was washed with MTBE: Hexane=2:1 (60 mL) and extracted with methylene chloride (50 mL×4). The methylene chloride extract was concentrated; the residue was dissolved in acetonitrile (48 mL) and treated with cold water (34 mL) over 20 min while stirring in an ice-water bath. The bottom layer was separated and partitioned between methylene chloride (150 mL) and aqueous (1:1) brine solution (160 mL). The organic layer was dried (anhydrous Na₂SO₄) and concentrated to give product (11.7 g) as a grey solid ³¹P NMR (CDCl₃, 121.5 MHz): δ=27.7-25.8 (m). This product was used in next step directly without further purification

To a stirring solution of above product (11.2 g) in 18 mL of methylene chloride at 0° C., pyrrole (2.85 mL, 41.1 mmol) was added followed by dichloroacetic acid (3.2 mL, 38.4 mmol) over 5 min. After stirring at 0° C. for 40 min, the reaction mixture was quenched by slow addition of saturated sodium bicarbonate (40 mL). The mixture was diluted with 20 mL of methylene chloride, the aqueous layer was separated and extracted with methylene chloride (40 mL) and the combined methylene chloride extract were dried (anhydrous Na₂SO₄) and concentrated. The residue was dissolved in 28 mL of methylene chloride, and treated sequentially with a solution A (MTBE: Hexane=1:1, 28 mL) over 10 min, Celite (16.8 g), and additional portion of the solution A (28 mL) over 20 min. The mixture was filtered; the solid was washed with solution A (40 mL) and MTBE (40 mL) and extracted with methylene chloride (4×50 mL). The methylene chloride extract was concentrated and purified with a short silica gel column. The column was eluted with methylene chloride. After concentration, product (8.7 g) was obtained as grey solid. Yield: 66%. ³¹P NMR (CDCl₃, 121.5 MHz): δ=27.8-26.3 (m).

### Example 23

### Preparation of HO-Gp(s)Tp(s)Gp(s)Ap(s)Gp(s)Tp(s)Gp(s)Ap(s)Gp(s)Tp(s)Gp(s)A-Lev (SEQ. ID NO: 1)

A mixture of triethylammonium salt of DMTr-Gp(s)Tp(s)Gp(s)Ap(H) (6.85 g, 2.64 mmol) and HO-Gp(s)Tp(s)Gp(s)Ap(s)Gp(s)Tp(s)Gp(s)A-Lev (8.2 g, 1.81 mmol) was rendered anhydrous by evaporation with pyridine. To the residue, anhydrous pyridine (14 mL) was added, the resulting solution was stirred under N₂ at 0° C. and treated with pivaloyl chloride (0.8 mL, 6.48 mmol) which was added over 3 min. The cold bath was removed and the mixture was stirred at ambient temperature for 30 min. The mixture was cooled to 0° C., and a solution of N-propionyloxymethylthio-N-methyl methanesulfonamide (1.23 g, 5.40 mmol) in anhydrous methylene chloride (2.0 mL) was added, followed by N N-diisopropylethylamine (0.56 mL, 3.24 mmol). After stirring at ambient temperature for 1 hour, the reaction mixture was diluted with 250 mL of methylene chloride and was washed with cold semi-saturated sodium bicarbonate (250 mL). The organic layer was dried (anhydrous Na₂SO₄) and concentrated. The residue was co-evaporated with 50 mL of toluene, dissolved in 43 mL of methylene chloride, and treated sequentially with a solution A (MTBE: Hexane=1:1, 43 mL) over 20 min, Celite (19.4 g) and an additional portion of the solution A (43 mL) over 30 min. After stirring for 30 min, the mixture was filtered, the solid was washed with a mixture made of the solution A and CH₂Cl₂ in the ratio of 4:1 (100 mL×2). The solid was extracted with methylene chloride (100 mL×4), and the extract was concentrated. The residue was dissolved in 65 mL of acetonitrile and treated with cold water (46 mL) added over 20 min. The bottom organic layer was separated and partitioned between methylene chloride (200 mL) and aqueous (1:1) brine solution (200 mL). The organic layer was dried (anhydrous Na₂SO₄) and concentrated to give product (12.8 g) as yellow foam. ³¹P NMR (CDCl₃, 121.5 MHz): δ=30.0-24.8 (m).

To a stirred solution of the above product (12.8 g) in 20 mL of methylene chloride at 0° C., pyrrole (2.64 mL, 38.0 mmol) was added followed by dichloroacetic acid (3.0 mL, 36.0 mmol). After stirring further at 0° C. for 30 min, the reaction mixture was diluted with 200 mL of methylene chloride, followed by a slow addition of saturated sodium bicarbonate (100 mL). The organic layer was separated, dried (Na₂SO₄) and concentrated. The residue was dissolved in 40 mL of methylene chloride, and treated sequentially with a solution A (MTBE: Hexane=1:1, 40 mL) added over 10 min, Celite (20 g) and additional portion of the solution A (80 mL) added over 20 min. The mixture was filtered and the solid was washed with a solvent mixture made from the solution A and CH₂Cl₂ in the ration of 5:1 (120 mL). The solid was extracted with methylene chloride (100 mL×4) and the extract concentrated. The residue was dissolved in 70 mL of acetonitrile and treated with cold water (49 mL) added over 30 min. The bottom organic layer was separated and partitioned between methylene chloride (150 mL) and aqueous (1:1) brine solution (150 mL). The organic layer was dried (Na₂SO₄) and concentrated to give product (9.2 g) as off-white solid. Yield: 75%. This product was used for next step directly. ³¹P NMR (CDCl₃, 121.5 MHz): δ=27.7-26.4 (m).

### Example 24

### Preparation of HO-Gp(s)Tp(s)Gp(s)Ap(s)Gp(s)Tp(s)Gp(s)Ap(s)Gp(s)Tp(s)Gp(s)Ap(s)Gp(s) Tp(s)Gp(s)A-Lev: (SEQ. ID NO: 2)

A mixture of triethylammonium salt of DMTr-Gp(s)Tp(s)Gp(s)Ap(H) (3.64 g, 1.4 mmol) and HO-Gp(s)Tp(s)Gp(s)Ap(s)Gp(s)Tp(s)Gp(s)Ap(s)Gp(s)Tp(s)Gp(s)A-Lev (6.1 g, 0.89 mmol) was rendered anhydrous by evaporation with pyridine. The residue was diluted with anhydrous pyridine (10 mL) and treated under N₂ at 0° C. with pivaloyl chloride (0.37 mL, 3.0 mmol) which was added slowly over 3 min. The cold bath was removed and the mixture was stirred at ambient temperature for 1 hour. The mixture was cooled to 0° C., and a solution of N-propionyloxymethylthio-N-methyl methanesulfonamide (568.3 mg, 2.5 mmol) in anhydrous methylene chloride (1.0 mL) was added, followed by N N-diisopropylethylamine (0.26 mL, 1.5 mmol). After stirring at ambient temperature for 1 hour, the reaction mixture was diluted with 120 mL of methylene chloride and washed sequentially with cold water (120 mL) and semi-saturated sodium bicarbonate (120 mL). The organic layer was dried (anhydrous Na₂SO₄) and concentrated. The residue was dissolved in 32 mL of methylene chloride, and treated sequentially with solution A (MTBE: Hexane=1:1, 32 mL) added over 20 min, Celite (16.0 g), and an additional portion of the solution A (64 mL) added over 30 min. After stirring for 30 min, the mixture was filtered, the solid was washed with a solvent mixture made of the solution A and CH₂Cl₂ in the ratio 5:1 (60 mL). The solid was extracted with methylene chloride (80 mL×4) and the extract was concentrated. The residue was dissolved in 40 mL of acetonitrile and treated with cold water (28 mL) added over 20 min with stirring. The bottom organic layer was separated and partitioned between methylene chloride (150 mL) and aqueous (1:1) brine solution (150 mL). The organic layer was dried (Na₂SO₄) and concentrated to give product (6.92 g) as yellow solid. ³¹P NMR (CDCl₃, 121.5 MHz): δ=27.6-26.3 (m). This product was used for next step without additional purification.

To a stirring solution of above product (6.92 g) in 10 mL of methylene chloride at 0° C., pyrrole (1.74 mL, 25.0 mmol) was added followed by dichloroacetic acid (2.0 mL, 24.0 mmol) over 2 min. After stirring further at 0° C. for 30 min, the reaction mixture was diluted with 100 mL of methylene chloride, followed by addition of saturated sodium bicarbonate (50 mL) slowly. The organic layer was separated and the aqueous layer was extracted with 50 mL of methylene chloride. The combined CH₂Cl₂ extracts were dried (anhydrous Na₂SO₄) and concentrated. The residue was dissolved in 32 mL of methylene chloride, and treated sequentially with solution A (MTBE: Hexane=1:1, 32 mL) added over 10 min, Celite (16 g) and an additional portion of the solution A (64 mL) added over 30 min. The mixture was filtered, the solid was washed with a solvent mixture made of the solution A and CH₂Cl₂ in the ratio of 5:1 (90 mL). The solid was extracted with methylene chloride (80 mL×4) and the extract was concentrated. The residue was dissolved in 40 mL of acetonitrile and treated with cold water (28 mL) which was added over 10 min while the mixture was stirred in an ice-bath. The bottom organic layer was separated and partitioned between methylene chloride (100 mL) and aqueous (1:1) brine solution (100 mL). The organic layer was dried (anhydrous Na₂SO₄) and concentrated to give product (5.8 g) as yellow solid. Yield: 71%. ³¹P NMR (CDCl₃, 121.5 MHz): δ=27.7-26.3 (m).

### Example 25

### Complete Deprotection of 5′-OH Fully-Protected Oligonucleotide Phosphorothioate HO-Gp(s)Tp(s)Gp(s)A-Lev

Fully-protected tetramer HO-Gp(s)Tp(s)Gp(s)A-Lev (1.38 g, 0.62 mmol) was rendered anhydrous by evaporation of added pyridine. To the residue, 1,2,4-triazole (192.7 mg, 2.79 mmol), 4 Å molecular sieve (1.5 g) and anhydrous pyridine (6.0 mL) were added. This mixture was stirred and cooled to 0° C. under N₂, and tert-butylamine (1.95 mL, 18.6 mmol) was added. The resulting mixture then was stirred at room temperature for 4 hours. The mixture was filtered and the molecular sieve was washed with pyridine (5 mL×2). The combined filtrate was concentrated to dryness. To the residue, syn-2-pyridinealdoxime (909 mg, 7.44 mmol) was added, followed by anhydrous acetonitrile (10 mL). The mixture was stirred and cooled to 0° C., and 1,8-diazabicyclo[5.4.0]undec-7-ene (1.67 mL, 11.2 mmol) was added. After stirring at room temperature for 15 hours, MTBE (50 mL) was added slowly over 10 min. After stirring further for 20 min, the top clear solution was decanted, and the residue was rinsed with ethyl acetate (20 mL). The residue was evaporated to remove the residual solvents and was dissolved in a mixture of 28% aqueous ammonia (10.0 mL) and 2-mercaptoethanol (0.5 mL). The resulting mixture was heated at 55° C. for 15 hours. After cooling down the mixture was added dropwise to a stirring mixture of isopropanol: THF=1:3 (80 mL) over 10 min. After stirring further for 20 min, the top clear solution was decanted, and the residue was rinsed with THF (20 mL). The residue was purified with a reversal C18 chromatography. The product obtained was applied to a column (8 cm×3 cm diameter) of Amberlite® IR-120 (plus) ion-exchange resin (sodium form). The column was eluted with water, and the desired fractions were combined and lyophilized to give the sodium form of the fully-deprotected oligonucleotide phosphorothioate of product 684 mg as white solid. Yield: 83%. ³¹P NMR (D₂O, 121.5 MHz): δ=55.4-54.6 (m).

### Example 26

### Synthesis of Fully Protected Dinucleotide Phosphorothioate as Shown in Scheme Below

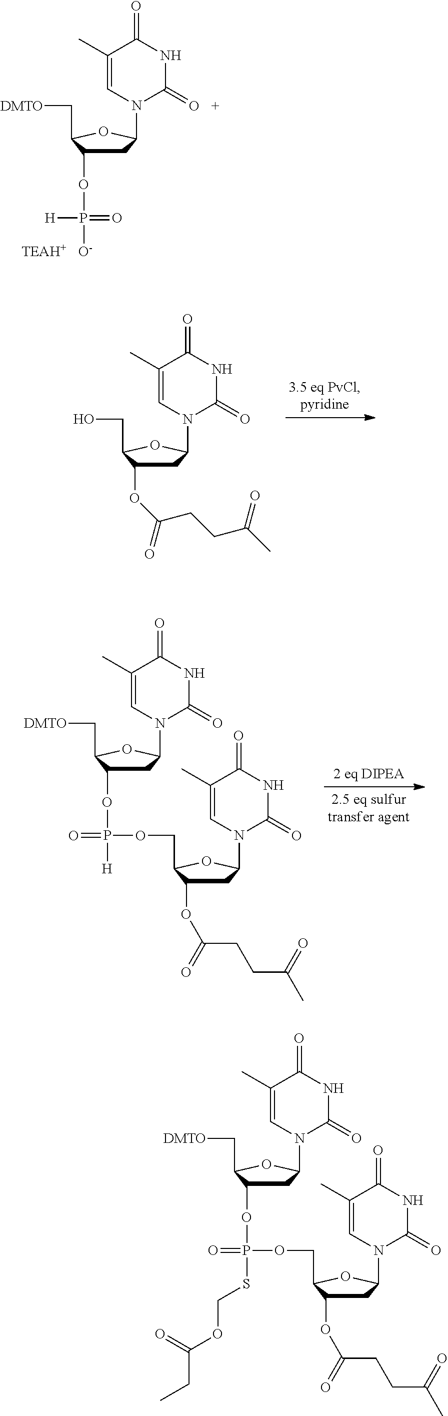

A solution of triethylammonium 5′-O-(4,4′-dimethoxytrityl)-thymidine-3′-H-phosphonate (425.9 mg, 0.6 mmol), 3′-O-levulinyl thymidine (170.2 mg, 0.5 mmol) and dry pyridine (10.0 mL) was rotary evaporated to dryness. The residue was redissolved in 10 mL of pyridine and rotary evaporated again to dryness. To the residue, molecular sieves (300 mg, activated, 3 Å) and anhydrous pyridine (5.0 mL) were added. The mixture was stirred at room temperature under N2, pivaloyl chloride (0.22 mL, 1.75 mmol) was added. After stirring at room temperature for 5 min, a solution of N-methyl-N-propionyloxymethylsulfanyl methanesulfonamide (284.1 mg, 1.25 mmol) in CH2Cl2 (1.0 mL) was added, followed by DIPEA (0.17 mL, 2.0 mmol). The resulting mixture was stirred at room temperature for 30 min. Ethyl acetate (30 mL) was added. The mixture was filtered and the filtrate was washed with water (15 mL), semi-saturated aqueous sodium bicarbonate (15 mL×2) and brine (15 mL). The organic layer was dried and evaporated to give 1.21 g of pale yellow oil. This crude was purified by silica gel chromatography (EtOAc/Acetone) to give product (389 mg) as colorless foam. Yield: 74.4%.

### Example 27

### Complete Deprotection of 5′-OH Fully-Protected Trimer as Shown in Scheme Below

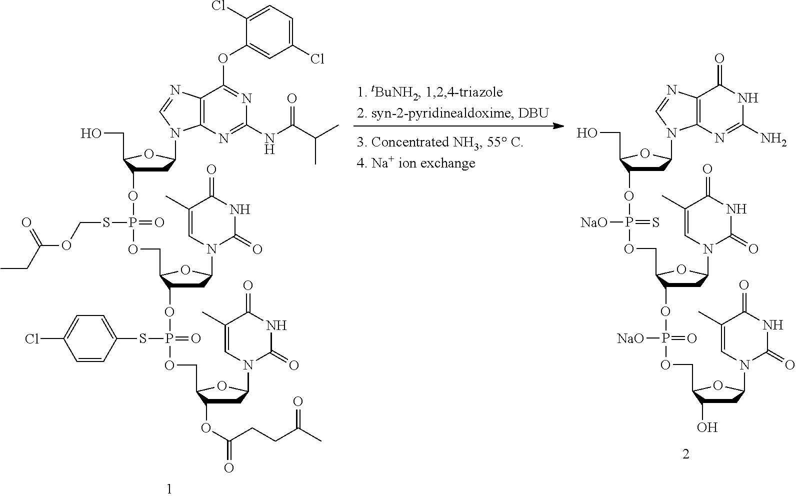

Fully-protected trimer 1 (7.09 g, 5.0 mmol) is rendered anhydrous by evaporation of added pyridine. To the residue, 1,2,4-triazole (690.7 mg, 10.0 mmol), 4 Å molecular sieve (2.0 g) and anhydrous pyridine (25.0 mL) are added. This mixture is stirred and cooled to 0° C. under N₂, and tent-butylamine (2.19 g, 30.0 mmol) is added. The resulting mixture then is stirred at room temperature for 4 hours. To the solution, syn-2-pyridinealdoxime (2.44 g, 20.0 mmol) is added, followed by 1,8-diazabicyclo[5.4.0]undec-7-ene (6.09 g, 40.0 mmol). After stirring at room temperature for 15 hours, the molecular sieve is filtered and washed with pyridine (10.0 mL). The filtrate is concentrated. The residue is dissolved in 28% aqueous ammonia (25.0 mL). The resulting solution is heated at 55° C. for 15 hours. After cooling down the mixture is concentrated and purified with a reversal C18 chromatography. The product obtained is applied to a column (100 g) of Amberlite® IR-120 (plus) ion-exchange resin (sodium form). The column is eluted with water, and the desired fractions are combined and lyphilized to give the desired product 2.

1. An oligonucleotide which comprises at least one internucleotide linkages comprising a P—S—R bond and at least two nucleosides, wherein R corresponds to the formula (I) 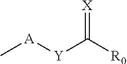
wherein A is a geminally substituted alkylene group, wherein X and Y are independently selected from the group consisting of S and O, and wherein R₀ is selected from the group consisting of optionally substituted carbon bonded organic residue, such as in particular optionally substituted alkyl or aryl, SRx, ORx and NRxRy wherein Rx and/or Ry are selected from H and organic residues and at least Rx is a substituent other than H.

2. The sulfurizing agent according to claims 1 wherein R is selected from the group consisting of a methyleneacyloxy group, a methylene carbamate group or a methylene carbonate group, and wherein R″ is a leaving group.

3. The sulfurizing agent according to of claim 1, wherein R corresponds to formula —CH₂—O—C(O)—R₀ wherein R₀ is a C1-C20, saturated, unsaturated, heterocyclic or aromatic, hydrocarbon residue.

4. The sulfurizing agent according to claim 2, wherein Rₓ is selected from the group consisting of lower alkyl or cycloalkyl (C1-C7), phenyl including substituted phenyl, and naphthyl groups.

5. The sulfurizing agent according to claim 1 wherein R″ is a sulfonamide group.

6. The sulfurizing agent according to claim 5 which corresponds to formula (III) 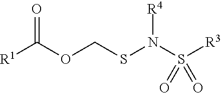
wherein R₁, R₃ and R₄ are independently a C1-C20, optionally unsaturated or aromatic, hydrocarbon residue.

7. The sulfurizing agent according to claim 1 wherein R″ is a dicarboxylamide.

8. A process for the synthesis of the sulfurizing agent according to claim 1, comprising (a) reacting a sulfuryl halide with a thioacetal of formula R—S—C(O)—R₂ wherein R is defined in claim 1 and wherein R₂ is an organic residue to produce an intermediate product of formula R—S—W, wherein W is halogen and, and (b) reacting said intermediate product with an N-sulfonyl compound or an N-acyl compound.

9. The process of claim 8 wherein the thioacetal is of formula R₁—C(O)—O—CH₂—S—C(O)—R₂ wherein R₁ and R₂ are independently a C1-C20 optionally unsaturated or aromatic hydrocarbon residue, and wherein said thioacetal is reacted with sulfuryl chloride to produce an intermediate product of formula R₁—C(O)—O—CH₂—S—Cl, wherein R₁ is independently a C1-C20, optionally unsaturated or aromatic, hydrocarbon residue.

10. The process according to claim 8 wherein in step (b) the intermediate is reacted with an N-sulfonyl compound of formula R₃—S(O)₂—NH—R₄, wherein R₃ and R₄ are independently organic residues.

11. A method for synthesizing an oligonucleotide which comprises using the sulfurizing agent according to claim 1 for sulfurizing at least one phosphorus internucleotide linkage of a precursor of said oligonucleotide.

12. The method according to claim 11 wherein the oligonucleotide comprises at least one internucleotide linkage comprising a P—S—R bond and at least two nucleosides, wherein R has the meaning given in claim 1 and corresponds to the formula (I) 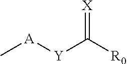
wherein A is a geminally substituted alkylene group, wherein X and Y are independently selected from S and O, and wherein R₀ is selected from the group consisting of optionally substituted carbon bonded organic residue, such as in particular optionally substituted alkyl or aryl, SRx, ORx and NRxRy wherein Rx and/or Ry are selected from H and organic residues and at least Rx is a substituent other than H.

13. The method according to claim 12 wherein R is selected from the group consisting of a methyleneacyloxy group, a methylene carbonate group, and a methylene carbamate group, or wherein R corresponds to formula —CH₂—O—C(O)—R₀ wherein R₀ is a C1-C20, saturated, unsaturated, heterocyclic or aromatic, hydrocarbon residue, or wherein R corresponds to a methylene carbamate group of formula —CH₂—O—C(O)—NRxRy wherein Rₓ and Ry are independently selected from alkyl or (hetero)aryl or Rₓ and Ry form together a 3 to 8 membered ring optionally containing an additional annular heteroatom selected from O, N and S, or wherein R corresponds to a methylene carbonate group of formula —CH₂—O—C(O)ORx wherein Rₓ is selected from the group consisting of optionally substituted alkyl cycloalkyl and (hetero)aryl groups.

14. The method according to claim 12 wherein Rₓ is selected from the group consisting of lower alkyl or cycloalkyl (C1-C7), phenyl including substituted phenyl, and naphthyl groups.

15. The method according to claim 11 for synthesizing an oligonucleotide which comprises using the sulfurizing agent according to claim 1 and at least one sulfurizing agent of formula R″—S—R′ wherein R′ is an organic residue other than the group R, and R″ is a leaving group.

16. The method according to claim 11 for synthesizing an oligonucleotide which comprises at least one internucleotide linkage comprising a P—S—R bond, at least one internucleotide linkage comprising a P—S—R′ bond and at least three nucleosides, wherein R corresponds to the formula (I) 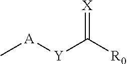
wherein A is a geminally substituted alkylene group, wherein X and Y are independently selected from the group consisting of S and O, and wherein R₀ is selected from the group consisting of optionally substituted carbon bonded organic residue, such as in particular optionally substituted alkyl or aryl, SRx, ORx and NRxRy wherein Rx and Ry are selected from H and organic residues and at least Rx is a substituent other than H, and
wherein R′ is an organic residue other than the group R.

17. The method according to claim 16 wherein R is selected from the group consisting of a methyleneacyloxy group, a methylene carbonate group, and a methylene carbamate group; and wherein R′ is an unsubstituted or substituted phenyl group.

18. The method according to claim 17, wherein the sulfurizing agent of formula R″—S—R′ corresponds to formula (V) 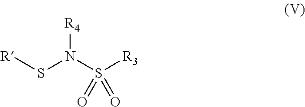
wherein R′ is selected from a group consisting of an aryl group and a heteroaryl group which is bonded to the S-atom through an annular carbon atom; and wherein R3 and R4 are independently a C1-C20, optionally unsaturated or aromatic, hydrocarbon residue.

19. The method according to claim 11 wherein the phosphorus internucleotide linkage is an H-phosphonate diester bond.

20. The method according to claim 19 which further comprises forming the H-phosphonate diester bond by coupling an H-phosphonate monoester salt with a protected nucleoside or oligonucleotide having a free hydroxy group.

21. The method according to claim 20 wherein the coupling is carried out in solution phase.

22. The method according to claim 11 for the manufacture of an oligonucleotide which comprises from 2 to 30 nucleotides.

23. The method according to claim 11 wherein the oligonucleotide contains nucleosides selected from the group consisting of ribonucleosides, 2′-deoxyribonucleosides, 2′-substituted ribonucleosides, 2′-4′-locked-ribonucleosides, 3′-amino-ribonucleosides, and 3′-amino-2′-deoxyribonucleosides.

24. The method according to claim 11 for the manufacture of a purified oligonucleotide further comprising a step wherein the oligonucleotide having at least one P—S—R linkage is purified by precipitation or extraction.

25. The method according to claim 12 comprising a step of producing a second oligonucleotide having at least one thiophosphate linkage, which further comprises cleaving at least one R group, from said oligonucleotide to produce a second oligonucleotide having at least one phosphorothioate linkage.

26. The method according to claim 16 further comprising a step of producing a fifth oligonucleotide having at least one phosphothioate diester linkage and at least one phosphodiester linkage, which comprises
(a) providing an oligonucleotide which comprises at least one internucleotide linkage comprising a P—S—R bond and at least three nucleosides, wherein R corresponds to the formula (I) 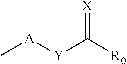
wherein A is a geminally substituted alkylene group, wherein X and Y are independently selected from the group consisting of S and O, and R₀ is selected from the group consisting of optionally substituted carbon bonded organic residue, such as in particular optionally substituted alkyl or aryl, SRx, ORx and NRxRy wherein Rx and/or Ry are selected from H and organic residues and at least Rx is a substituent other than H; and
(b) cleaving at least one R group, from said oligonucleotide to produce a fourth oligonucleotide having at least one thiophosphate diester linkage and,
(c) subsequently cleaving at least one R′ group, from said fourth oligonucleotide to produce a fifth oligonucleotide having at least one phosphothioate diester linkage and at least one phosphodiester linkage.

27. The method according to claim 25 wherein the cleavage is carried out in the presence of a sterically hindered base and of an activator.

28. The method according to claim 27 wherein the activator is 1,2,4-triazole or selected from other triazole and tetrazole derivatives.

29. The method according to claim 26 wherein the R′ group is cleaved by an oximate treatment.

30. The method according to claim 26, wherein the R group is cleaved in the human body or in the body of an animal.

31. An oligonucleotide which comprises at least one internucleotide linkage comprising a P—S—R bond and at least three nucleosides, wherein R corresponds to the formula (I) 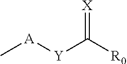
wherein A is a geminally substituted alkylene group, wherein X and Y are independently selected from the group consisting of S and O, and R₀ is selected from the group consisting of optionally substituted carbon bonded organic residue, such as in particular optionally substituted alkyl or aryl, SRx, ORx and NRxRy wherein Rx and/or Ry are selected from H and organic residues and at least Rx is a substituent other than H.

32. The oligonucleotide according to claim 31 which comprises at least one internucleotide linkage comprising a P—S—R bond.

33. The oligonucleotide according to claim 31 which comprises from 2 to 30 nucleotides.

34. The oligonucleotide according to claim 31 which contains nucleosides selected from the group consisting of ribonucleosides, 2′-deoxyribonucleosides, 2′-substituted ribonucleosides, 2′-4′-locked-ribonucleosides, 3′-amino-ribonucleosides, and 3′-amino-2′-deoxyribonucleosides.

35. The oligonucleotide according to claim 31 as a prodrug.


SEQUENCE LISTING (2 sequences)

SEQ ID NO: 1 - DNA, 12 bp, Artificial
  Synthetic Sequence
gtgagtgagtga

SEQ ID NO: 2 - DNA, 16 bp, Artificial
  synthesis exemple
gtgagtgagtgagtga